(19) United States
(12) Patent Application Publication
(10) Pub. No.: US 2007/0059697 A1
(43) Pub. Date: Mar. 15, 2007

(54) Determining cancer-linked genes and therapeutic targets using molecular cytogenetic methods

(75) Inventors: Jeffrey W. Strovel, North Potomac, MD (US); Colyn B. Cain, Bethesda, MD (US); Stephen K. Horrigan, Potomac, MD (US); Meena Augustus, Gaithersburg, MD (US)

Correspondence Address: CARELLA, BYRNE, BAIN, GILFILLAN, CECCHI,;STEWART & OLSTEIN, 5 BECKER FARM ROAD, ROSELAND, NJ, 07068, US

(73) Assignee: AVALON PHARMACEUTICALS, INC, Germantown, MD (US)

(21) Appl. No.: 10/553,098

(22) PCT Filed: Apr. 15, 2004
(86) PCT No.: PCT/US04/09289

§ 371 Date: Nov. 21, 2006

Related U.S. Application Data
(60) Provisional application No. 60/462,895, filed on Apr. 15, 2003.

(51) Int. Cl. C12Q 1/68; C12P 19/34

(52) U.S. Cl. 435/6; 435/91.2

(57) ABSTRACT

Methods for identifying potential therapeutic agents, such as anti-tumor agents, based on their modulation of the expression of specified genes, especially genes mapping to specific chromosomal regions, are disclosed. Also described are methods for diagnosing cancerous, or potentially cancerous, conditions as a result of the expression, or patterns of expression, of such genes, including detecting changes in levels of gene copy number and/or level of amplification of the said gene, or sets of genes, to detect and/or diagnose the cancer. Methods for detecting or determining functionally related genes, as well as methods for treating cancer based on targeting expression products of such genes, determining genes involved in the cancerous process and the success and/or response rates and survival statistics for cancer patients on treatment are encompassed by the invention. Also encompassed are methods involving determining the modulated expression of the genes in these regions of interest (ROIs) as pharmacodynamic/pharmacogenetic /surrogate markers and/or for patient profiling prior to accrual for clinical trials/treatments based on the identification of these genes as validated gene/drug targets in various cancer tissue types.

This application claims priority of U.S. Provisional Application Ser. No. 60/462,895, filed 15 Apr. 2003, the disclosure of which is hereby incorporated by reference in its entirety.

### FIELD OF THE INVENTION

The present invention relates to identification of genes whose disruption and/or change in expression is useful to distinguish cancerous from non-cancerous tissue and serve as potential therapeutic targets, pharmacodynamic/pharmacogenetic/surrogate and prognostic and diagnostic markers, and which genes are identified by high resolution Comparative Genomic Hybridization (CGH) and Spectral Karyotyping (SKY)/fluorescent in situ hybridization (FISH) analysis of DNA and chromosomes of various cancer cell lines and primary and metastatic tumor samples combined with gene expression analysis of these cells and tissues.

### BACKGROUND OF THE INVENTION

Chromosomal abnormalities have been identified in most cancer cells.

Conventional chromosome banding techniques allow for the detection of specific chromosomal defects in tumor cells but interpretation of the banding pattern is sometimes difficult, particularly when complex chromosomal rearrangements or subtle abnormalities are present. In recent years, new techniques, such as CGH and SKY, based on fluorescent in situ hybridization (FISH) (Pinkel et al., Proc Nat Acad Sci USA 85:9138-42 (1988)) have been developed to overcome the limitations of conventional chromosome banding. CGH measures intensities of fluorescently labeled tumor DNA and normal DNA following hybridization to normal chromosomes (Kallioniemi et al., Science 258:818-21 (1992)). Gain or loss of copy number of a particular chromosome or chromosome region in the tumor DNA is determined by the relative intensity of a fluorescence ratio. SKY utilizes a cocktail of chromosome probes, fluorescently labeled to specify each chromosome, which is hybridized to tumor chromosomes in an effort to identify numerical and structural abnormalities in the tumor cell (Schröck et al., Science 273:494-7 (1996)). CGH and SKY have been used to identify chromosomal regions that harbor genes significant to the process of tumor initiation or progression.

### BRIEF SUMMARY OF THE INVENTION

In one aspect the present invention relates to a set of genes that have been localized within human chromosomal regions of interest (ROI) that have been identified by molecular cytogenetic techniques.

In one aspect, the present invention relates to a method for diagnosing cancer in a mammal, especially a human patient, comprising determining amplification of a gene in the genome of a mammal wherein said gene is a gene of Table 1.

In a preferred embodiment thereof, the cancer is a member selected from breast cancer, colon cancer, lung cancer, prostate cancer, ovarian cancer, pancreatic cancer, cervical cancer and kidney cancer.

In another preferred embodiment thereof, 3. The method of claim 1 wherein said gene of Table 1 is a gene that encodes the same gene product as a polynucleotide selected from the polynucleotides of SEQ ID NO: 1-805 and 855-923.

In another embodiment, the present invention relates to a method for diagnosing cancer or a pre-cancerous condition in a mammal, comprising:

(a) obtaining a cell or tissue sample from a mammal, especially a human patient, suspected of having cancer or a pre-cancerous condition and determining for said sample the gene copy number of a gene of Table 1;

(b) comparing said gene copy number of step (a) to the gene copy number of the same gene from a sample of a corresponding cell or tissue from a mammal of the same species not having cancer of the type being diagnosed whereby a higher gene copy number determined in step (a) relative to that in step (b) indicates the presence of a cancer or pre-cancerous condition in the mammal of step (a) and results in a diagnosis of cancer or a pre-cancerous condition in said mammal.

In a preferred embodiment of the methods of the invention, said molecule is a member selected from an antisense DNA, an antisense RNA, a ribozyme and an siRNA.

In another embodiment, the present invention relates to a method for identifying an agent having therapeutic activity in a human patient in need of said therapeutic activity, comprising:

(a) determining in a sample from a patient the level of a gene product encoded by a gene of Table 1 prior to administering a test compound to said patient;

(b) administering said test compound to said patient;

(c) determining in a sample from said patient the level of a gene product encoded by the same the gene as in step (a)

wherein a decrease in the level of said gene product in step (c) relative to step (a) identifies said test compound as an agent having therapeutic activity.

In a further embodiment, the present invention relates to a method for identifying an antineoplastic agent, comprising:

(a) contacting a test compound with a cell that expresses a gene of Table 1; and

(b) determining a change in gene expression as a result of said contacting;

whereby said change in said gene expression identifies said test compound as an antineoplastic agent.

The present invention also relates to a method for determining the cancerous status of a cell, comprising determining elevated expression in said cell of a gene of Table 1 wherein elevated expression of said gene indicates that said cell is cancerous.

In an additional embodiment, the present invention relates to a method for identifying a compound as an anti-neoplastic agent, comprising:

(a) contacting a test compound with a polypeptide encoded by a gene of Table 1,

(b) determining a change in a biological activity of said polypeptide due to said contacting,

wherein a change in activity identifies said test compound as an agent having antineoplastic activity.

In a preferred embodiment of the foregoing, the polypeptide is an enzyme selected from kinase, protease, peptidase, phosphodiesterase, phosphatase, dehydrogenase, reductase, carboxylase. transferase, deacetylase and polymerase.

The present invention also relates to a method for identifying an anti-neoplastic agent comprising contacting a cancerous cell with a compound found to have anti-neoplastic activity in other the methods of the invention under conditions promoting the growth of said cell and detecting a change in the activity of said cancerous cell.

The present invention further relates to a method for treating cancer comprising contacting a cancerous cell with an agent having affinity for an expression product of a gene of Table 1 and in an amount effective to cause a reduction in cancerous activity of said cell.

The present invention also contemplates a method for monitoring the progress of cancer therapy in a patient comprising monitoring in a patient undergoing cancer therapy the expression of a gene of Table 1.

In addition, the present invention encompasses a method for determining the likelihood of success of cancer therapy in a patient, comprising monitoring in a patient undergoing cancer therapy the expression of a gene of Table 1 wherein a decrease in said expression prior to completion of said cancer therapy is indicative of a likelihood of success of said cancer therapy.

In another embodiment, the present invention relates to a method for producing test data with respect to the anti-neoplastic activity of a compound comprising:

(a) identifying a test compound as having anti-neoplastic activity using other methods of the invention;

(b) producing test data with respect to the anti-neoplastic activity of said test compound sufficient to identify the chemical structure of said test compound.

Additionally, the present invention encompasses a method for determining the progress of a treatment for cancer in a patent afflicted therewith, following commencement of a cancer treatment on said patient, comprising:

(a) determining in said patient a change in expression of one or more genes of Table 1; and

(b) determining a change in expression of said gene compared to expression of said one or more determined genes prior to said cancer treatment;

wherein said change in expression indicates progress of said treatment thereby determining the progress of said treatment.

### SEQUENCE LISTING ON CD-ROM ONLY

The sequences disclosed herein as SEQ ID NO: 1-923 in the sequence listing are contained on compact disc (CD-ROM) only, which accompanies this application and the contents of said CD-ROMs are hereby incorporated by reference in their entirety. These sequence numbers also appear in Table where all sequences are referred to as consecutive serial numbers for reference purposes only.

### DETAILED SUMMARY OF THE INVENTION

The present invention relates to a set of genes that are amplified and/or over-expressed genes in cancer cell lines and have been localized to various chromosomal regions of interest. These genes have been identified through a combination of CGH, SKY, expression analysis and Reverse Transcriptase-Polymerase Chain Reaction (RT-PCR). Such genes are both markers and potential therapeutic targets for cancer, in particular breast, colon, lung and prostate malignancies. In addition, the amplified nature of such genes provides a means of diagnosing a cancerous condition, or predisposition to a cancerous conditions, by determining the amplification of one or more of such genes in a patient afflicted with, or predisposed toward, or otherwise at risk of developing, cancer.

In accordance with the present invention, a number of genes have been localized to a chromosomal regions of interest as identified in Table 1 (serial number 1-229 (breast), 230-440 (colon), 441-656 (lung) and 657-805 (prostate), serial number 806-923 (transcript or protein)). The invention also includes any subsets of these. As described herein, these sequences include DNA sequences of SEQ ID NO: 1-805, transcripts with the sequences of SEQ ID NO: 855-923, and proteins/polypeptides with amino acid sequences of SEQ ID NO: 806-854.

Briefly, the procedures used to identify the genes disclosed herein may be summarized as follows:

For CGH analysis, based on detailed molecular cytogenetic characterizations, the following data sets are generated, which may include regions reported in the public domain as well as unique regions not previously known.
1. A map of chromosomal regions involved in consistent, recurrent and high level genomic gains (i.e., amplifications) for a representative cancer cell line or tumor type (e.g. colon, prostate, breast and lung) that can be recognized as a pattern/signature for a given tumor type.
2. A map of chromosomal regions containing genomic losses (i.e., deletions) in each tumor type and individual cell line to be examined.
3. Levels of intensities of gains and losses categorized for entry into a database.
4. A comparison of the patterns of gains and losses between the clinical samples (e.g. colon xenografts) and cell lines (e.g., colon) of matched Stages and Grades.
5. A comparison of the patterns of gains and losses between primary prostate tumor cell lines (e.g., CPDR lines) and metastatic prostate tumor cell lines (e.g., DU 145, PC3 and LNCaP).

In accordance with the present invention, for SKY analysis, data sets were generated according to the following steps:
1. Identification and development of a database of novel chromosomal rearrangements in epithelial cancer cell lines.
2. Identification of novel translocations involving specific chromosomes or chromosomal regions
3. Reconciliation of SKY and CGH analysis on the same cell line as a verification of the combined findings.

Combining genomic DNA analysis of gains and losses in the tumor cell lines/clinical samples with cDNA expression analysis from matched tumor types displayed on a genome template from the Golden Path genome browser using a Spotfire™ analysis tool:
1. A pattern of gene expression on a U-95 Affymatix chip set obtained via the Gene Logic database was used to generate differential gene expression profiles between samples sets containing normal and. malignant tissues from colon, prostate, lung, breast and various cell lines.
2. A Spotfire™ visualization tool was developed that allowed the generation of a list of all the genes that are present in the Golden Path within the clustered regions of gains/losses for each cell type/tumor type to generate the gene sets to include in the HITS platform
3. The following algorithm was employed:
   i) Match chromosomal regions of amplification/gains defined by CGH with the location of genes/ESTs on an Affymatix chip as mapped to a Golden Path genome template.
   ii) Identify genes/ESTs over-expressed in tumor tissue compared to normal tissue in said chromosomal regions using the Gene Logic database.
   iii) Compile data on cell lines of a particular tumor type and different tumor types showing clusters of genomic gains and losses at certain chromosomal regions.
   iv) Pick BACs that span the chromosomal regions consistently gained and containing over-expressed genes in an effort to positionally clone novel cancer genes (oncogenes and genes in relevant pathways)
   v) Validate the identified genes by
      A) Picking STS markers that identify the gene sequence and quantify the relative copy number in genomic DNA and RNA across a panel of tumor cell lines.
      B) Develop probes for FISH on chromosomes from tumor cell lines and primary tumor tissue micro-arrays.
4. The expression data from tumor cell lines that have undergone SKY/CGH analysis was used to pick candidate genes to validate as individual targets in functional genomic assays and in-vivo assays and for use in the transcriptional assay platform.

In accordance with the present invention, over-expression of cellular genes is conveniently monitored in model cellular systems using cell lines (such as is used in the example below), primary cells, or tissue samples maintained in growth media. For different purposes, these may be treated with compounds at one or more different concentrations to assay for modulating agents. Thus, cellular. RNAs were isolated from the cells or cultures as an indicator of selected gene expression. The cellular RNAs were then divided and subjected to analysis that detected the presence and/or quantity of specific RNA transcripts, which transcripts were then amplified for detection purposes using standard methodologies, such as reverse transcriptase polymerase chain reaction (RT-PCR). The levels of specific RNA transcripts, including their presence or absence, were determined. When used for identification of modulating agents, such as ant-neoplastic agents, a metric is derived for the type and degree of response of the treated sample compared to control samples.

In accordance with the foregoing, the genes identified as being amplified and/or over-expressed, which can include increased copy number thereof, in cancerous cells are localized in chromosomal regions of interest as identified in Table 1 (serial number 1-229 (breast), 230440 (colon), 441-656 (lung) and 657-805 (prostate); for polypeptide SEQ ID NOs, see Table 1, serial number 806-923 (transcript or protein)).

These genes may be utilized to characterize, the cancerous, or non-cancerous, status of cells, or tissues. The methods of the invention may be used with a variety of cell lines or with primary samples from tumors maintained in vitro under suitable culture conditions for varying periods of time, or in situ in suitable animal models.

The genes disclosed herein are expressed at levels in cancer cells that are different from the expression levels in non-cancer cells. These genes as identified in Table 1 are amplified in cancer cells relative to non-cancer cells of corresponding tissues, especially breast cancer, colon cancer, lung cancer, prostate cancer, ovarian cancer, pancreatic cancer, cervical cancer and kidney cancer.

In accordance with the foregoing, the present invention relates to a method for diagnosing cancer in a mammal, comprising determining amplification of a gene in the genome of a mammal wherein said gene is a gene of Table 1.

In a preferred embodiment thereof, said gene of Table 1 is a gene that encodes the same gene product as a polynucleotide selected from the polynucleotides of SEQ ID NO: 1-805 and 855-923. In a further preferred embodiment, said mammal is a human patient.

The present invention is also directed to a method for diagnosing cancer or a pre-cancerous condition in a mammal, preferably a human patient, comprising:

(a) obtaining a cell or tissue sample from a mammal suspected of having cancer or a pre-cancerous condition and determining for said sample the gene copy number of a gene of Table 1;

(b) comparing said gene copy number of step (a) to the gene copy number of the same gene from a sample of a corresponding cell or tissue from a mammal of the same species not having cancer of the type being diagnosed

whereby a higher gene copy number determined in step (a) relative to that in step (b) indicates the presence of a cancer or pre-cancerous condition in the mammal of step (a) and results in a diagnosis of cancer or a pre-cancerous condition in said mammal.

In specific embodiments, the cancer to be diagnosed is one or more of breast cancer, colon cancer, lung cancer, prostate cancer, ovarian cancer, pancreatic cancer, cervical cancer and kidney cancer.

Preferably, the gene of Table 1 is a gene that encodes the same gene product as a polynucleotide of SEQ ID NO: 1-805 and 855-923.

The present invention is also directed to a method of inhibiting cancer, or a pre-cancerous condition, in a mammalian cell, comprising contacting said cell with a molecule that inhibits function of a gene of Table 1. Preferably, the gene of Table 1 is a gene that encodes the same gene product as a polynucleotide of SEQ ID NO: 1-805 and 855-923. In a specific embodiment thereof, said molecule inhibits gene function by binding to said gene. In other embodiments, the molecule inhibits gene function by binding to an RNA encoded by said gene or inhibits gene function by binding to polypeptide encoded by said gene. Preferably, the molecule is a member selected from an antisense DNA, an antisense RNA, a ribozyme and an siRNA. Also preferred is where the cancer is a member selected from breast cancer, colon cancer, lung cancer, prostate cancer, ovarian cancer, pancreatic cancer, cervical cancer and kidney cancer.

The invention contemplates that such contacting occurs in vivo.

The invention also relates to a method for identifying an agent having therapeutic activity in a human patient in need of said therapeutic activity, comprising:

(a) determining in a sample from a patient the level of a gene product encoded by a gene of Table 1 prior to administering a test compound to said patient;

(b) administering said test compound to said patient;

(c) determining in a sample from said patient the level of a gene product encoded by the same the gene as in step (a)

wherein a decrease in the level of said gene product in step (c) relative to step (a) identifies said test compound as an agent having therapeutic activity.

Preferably, said therapeutic activity is anticancer activity and said cancer is one or more of breast cancer, colon cancer, lung cancer, prostate cancer, ovarian cancer, pancreatic cancer, cervical cancer and kidney cancer.

Also preferred is where said gene product is an RNA or a polypeptide, especially where an activity of the polypeptide is determined, preferably an enzyme activity. In specific embodiments, said gene of Table 1 is a gene that encodes the same gene product as a polynucleotide of SEQ ID NO: 1-805 and 855-923, as well as where said molecule is a member selected from an antisense DNA, an antisense RNA, a ribozyme and an siRNA.

The present invention also relates to a method for identifying an antineoplastic agent, comprising:

(a) contacting a test compound with a cell that expresses a gene of Table 1; and

(b) determining a change in gene expression as a result of said contacting;

whereby said change in said gene expression identifies said test compound as an antineoplastic agent.

Most preferred is where the change in expression is a decrease in expression. The contacting may occur in vivo. Also preferred is where said gene of Table 1 encodes the same gene product as a polynucleotide of SEQ ID NO: 1-805 and 855-923 and where said molecule is a member selected from an antisense DNA, an antisense RNA, ribozyme, an siRNA, a small organic molecule and an antibody.

The present invention also relates to a method for determining the cancerous status of a cell, comprising determining elevated expression in said cell of a gene of Table 1 wherein elevated expression of said gene indicates that said cell is cancerous. Preferably, wherein said elevated expression is an elevated copy number of the gene and wherein said gene of Table 1 encodes the same gene product as a polynucleotide of SEQ ID NO: 1-805 and 855 -923.

The present invention further relates to a method for identifying a compound as an anti-neoplastic agent, comprising:

(a) contacting a test compound with a polypeptide encoded by a gene of Table 1,

(b) determining a change in a biological activity of said polypeptide due to said contacting,

wherein a change in activity identifies said test compound as an agent having antineoplastic activity.

Preferably, said gene of Table encodes the same gene product as a polynucleotide of SEQ ID NO: 1-805 and 855-923.

In a preferred embodiment, the change in biological activity is a decrease in biological activity.

In another preferred embodiment, the biological activity is an enzyme activity, such as where the enzyme is one selected from the group kinase, protease, peptidase, phosphodiesterase, phosphatase, dehydrogenase, reductase, carboxylase. transferase, deacetylase and polymerase.

Assays for these enzymes are available, such as for phosphodiesterases (the most pharmacologically relevant phosphodiesterases are those that hydrolyze cyclic nucleotides (see, for example, cAMP and cGMP assays available from Perkin-Elmer, as well as Estrade et al., Eur. J. Pharmacol. 352:2-3, 157-163 (1998)).

Protein phosphatases remove phosphate residues from proteins. Most tests of their activity use the same assays as for protein kinases. A non-radioactive phosphatase assay system is available from Promega Biotech.

The therapeutically most relevant dehydrogenases oxidize or reduce small molecular weight metabolites, esp. steroid hormones, or that generally use or generate NAD or NADP (see: Haeseleer et al., J. Biol. Chem., 273:21790-21799 (1998)). A commercial assay is available from Cayman Chemical (at www.caymanchem.com).

Gamma-carboxylases are important enzymes in the blood coagulation process. The main assay protocols use synthetic peptides (see: Ulrich et al., J. Biol. Chem., 263:9697-9702 (1988); Begley et al., J. Biol. Chem., 275:36245-36249 (2000)).

In highly preferred embodiments, the kinase is one of a protein kinase, a serine or threonine kinase, or a receptor tyrosine protein kinase. Where the polypeptide encoded by a gene of the invention is a protein kinase, especially involving tyrosine kinase, various assays for activity are available. Protein kinases add phosphate groups to serine, threonine or tyrosine residues on proteins, most commonly measured with phospho-serine, threonine, or tyrosine-specific antibodies, or generation of radiolabeled substrate, or consumption of ATP, or phosphorylation of (synthetic) small peptides, or measuring downstream enzyme activity and gene transcription. Such assays are commercially available. (See, for example, the tyrosine kinase assay from Roche Molecular Biochemicals). Assays for serine/threonine kinases are also available at Chromagen.com, Upstate Biotechnology, Inc. (Lake Placid, N.Y., and at upstatebiotech.com) and from Applied BioSystems (Foster City, Calif. (home.appliedbiosystems.com)).

In other specific embodiments, the protease is a serine protease, cysteine protease or aspartic acid protease, or the transferase is a methyltransferase, preferably a cytosine methyltransferase or an adenine methyltransferase, or the deacetylase is a histone deacetylase, or the carboxylase is a γ-carboxylase, or the peptidase is a zinc peptidase, or the polymerase is a DNA polymerase or an RNA polymerase.

Proteases degrade proteins, un-specifically or at specific sites. Almost all pharmacologically relevant ones have very narrowly defined specific substrates, and their activity is most often measured by directly measuring cleavage product or generation of (fluorescent) light after cleavage of synthetic substrates. Assays are available for serine proteases (Calbiochem, Palo Alto, Calif., and see Berdichevsky et al., J. Virol. Methods, 107:245-255 (2003), for systeine proteases (See: Schulz et al., Mol. Pathol., 51:222-24 (1998) and Seizer et al., PNAS, 96:11015-11022 (1999)), for aspartic acid proteases (Geno Tech, Inc. at www.genotech.com) and for zinc peptidases (see Evans et al., J. Biol. Chem., 278:23180-23186 (2003)).

Both (regulatory) DNA-methylases and (biosynthetic) protein methylases that are drug targets. (See: Jonassen and Clarke, J. Biol. Chem., 275:12381-12387 (2000); Jackson et al., Nature, 416:556-560 (2002)).

Most HDAC (histone deacetylase) assays use colorimetric or fluorometric (synthetic) substrates. Standard assays are for binding, especially molecular size changes, blocking a specific site, and effects on transcription or downstream reactions (if DNA or RNA is the direct target of a drug). A commercial assay is available from Vinci Biochem (at www.vincibiochem.it).

In another specific embodiment, the biological activity is a membrane transport activity, preferably wherein the polypeptide is a cation channel protein, an anion channel protein, a gated-ion channel protein or an ABC transporter protein. Drug effects on the activity of transporter and channel proteins are screened by measuring increase or decrease of the ((radio-)labeled) transported entity inside or outside the cell, in cell-based assays, ATP consumption (in the case of ATPases), or changes in cell membrane potential. Assays employing such proteins are available, such as for ABC transporter (see: Marcil et al., Lancet, 354:1341-46 (1999) and for ion channels (from Evotec OAI, at www.evotecoai.com and from PharmaLinks, at www.pharmalinks.org/research/cellsignalling).

In one embodiment, the polypeptide is an integrin (the signal transduction pathways elicited by the integrins are slow and not very well characterized, hence most assays are either just binding assays or measure downstream biological phenomena (such as migration, invasion, etc.) (See: Ganta et al., Endocrinology, 138:3606-3612 (1997); Sim et al., J. Biomed. Mater. Research, 68A:352-359 (2004); and Weinreb et al., Anal. Biochem., 306:305-313 (2002)), or a Cytochrome P450 enzyme (almost all cytochrome assays require knowledge of what the substrate is and measure conversion of substrate (free or (radio-)labeled) or generation of product; useful C¹⁴-labeled substrates are available from Amersham Biosciences at wwwl.amershambiosciences.com), or a nuclear hormone receptor (Assays available from Discoverx, Fremont, Calif., such as an estrogen assay; also see Rosen et al., Curr. Opin. Drug. Discov. Devel., 6:224-30 (2003)).

In one preferred embodiment, the biological activity is a receptor activity, preferably where the receptor is a G-protein-coupled receptor (GPCR).

GPCRs are transmembrane proteins that wind 7 times back and forth through a cell's plasma membrane with a ligand binding site located on the outside of the membrane surface of the cell and the effector site being present inside the cell. These receptors bind GDP and GTP. In response to ligand binding, GPCRs activate signal transduction pathways which induce a number of assayable physiological changes, e.g., an increase in intracellular calcium levels, cyclic-AMP, inositol phosphate turnover, and downstream gene transcription (directly or via reporter-assays) along with other translocation assays available for measuring GPCR activation when the polypeptide encoded by a gene of the invention is a GPCR. Thus, such proteins work through a second messenger. The result is activation of CREB, a transcription factor that stimulates the production of gene products. One useful assay is the so-called BRET2/arrestin assay, useful in screening for compounds that interact with GPCRs. (See: Bertrand et al, J. Recept. Signal Transduct Res., 22:533-41 (February-November 2002)). In addition, numerous assays are commercially available, such as the Transfluor Assay, available from Norak Biosciences, Inc. (www.norakbio.com) or ArrayScan and KineticScan, both from Cellomics, or assays from CyBio (Jena, Germany).

Assays useful with the invention are usually set up to screen for agonists or antagonists after adding ligand, but effects on most of these parameters can be measured whether or not the ligand for the receptor is known. Such assays may involve radioligand-binding assays. Activation of the majority of GPCRs by agonists leads to the interaction of beta-arrestin (a protein that is involved in receptor desensitization and sequestration) with the receptor, which is measurable by fluorescence energy transfer

The disclosure of all journal articles, or other publications, referred to herein are hereby incorporated by reference in their entirety.

In one embodiment, the polypeptide is in a solution or suspension and contact with the test compound is by direct contact between the test compound and the protein molecule. Alternatively, the polypeptide may be in a cell and the test compound may have to diffuse into the cell in order to contact the polypeptide. In an alternative embodiment, the test compound may be contacted with a cell that contains or expresses the polypeptide but the test compound may have no direct contact with the polypeptide. In stead, the test compound may act to induce production and/or activity of a different compound, such as an intracellular second messenger that serves to contact the polypeptide and modulate or change the biological activity of this polypeptide.

In accordance with the foregoing, the method of the present invention includes cancer modulating agents that are themselves either polypeptides, or small chemical entities, that affect the cancerous process, including initiation, suppression or facilitation of tumor growth, either in vivo or ex vivo. Such agents may also be antibodies that react with one or more polypeptides encoded by genes as disclosed herein, preferably polypeptides comprising any one of the amino acid sequences of SEQ ID NO: 806-854.

Because the genes disclosed herein are over-expressed and relate to the cancerous condition of a cell, successful anti-neoplastic activity will commonly be exhibited by agents that reduce the expression of said genes as identified in Table 1. In one embodiment thereof, the change in expression is a decrease in copy number of the gene or genes under study. In accordance therewith, said change in gene copy number is conveniently determined by detecting a change in expression of messenger RNA encoded by said gene sequence. In another preferred embodiment, expression is determined for more than one such gene, such as 2, 5, 10 or more of the genes.

Other methods useful in measuring a change in expression of the genes disclosed herein include measuring a change in the amount or rate of synthesis of a polypeptide encoded by said gene, preferably a decrease in synthesis of said polypeptide. Most preferably, the polypeptide comprises an amino acid sequence highly homologous to a sequence for genes as identified in Table 1 (SEQ ID NO: 1-923).

The methods of the invention can thus be utilized to identify anti-neoplastic agents useful in treatment of cancerous conditions. Such activity can be further modified by first identifying such an agent using an assay as already described and further contacting such agent with a cancerous cell, followed by monitoring of the status of said cell, or cells. A change in status indicative of successful anti-neoplastic activity may include a decrease in the rate of replication of the cancerous cell(s), a decrease in the total number of progeny cells that can be produced by said cancerous cell(s), or a decrease in the number of times said cancerous cell(s) can replicate, or the death of said cancerous cell(s).

Anti-neoplastic agents may also be identified using recombinant cells suitably engineered to contain and express the cancer-related genes disclosed herein. In one such embodiment, a recombinant cell is formed using standard technology and then utilized in the assays disclosed herein. Methods of forming such recombinant cells are well known in the literature. See, for example, Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference.

The present invention also relates to a method for detecting the cancerous status of a cell, comprising detecting elevated copy number and/or expression in said cell of at least one gene that maps to the chromosomal region of interest as identified in Table 1 (SEQ ID NO: 1-923). Such elevated expression may be readily monitored by comparison to that of otherwise normal cells having the same genes. Elevated expression of these genes is indicative of the cancerous state. This includes a gene corresponding to a polynucleotide that comprises a nucleotide sequence as identified in Table 1 (SEQ ID NO: 1-923). Such elevated expression, including increased copy number, may be the expression of more than one such gene.

The present invention also relates to a method for detecting a cancer-linked gene comprising the steps of contacting a compound identified as having gene modulating activity for a gene corresponding to a polynucleotide that comprises a nucleotide sequence as identified in Table 1 (SEQ ID NO: 1-923) with a cell expressing a test gene and detecting modulation, such as decreased activity, of such test gene relative to when said compound is not present thereby identifying said test gene as a cancer-related gene. In preferred embodiments, the gene determined by said method is an oncogene, or cancer facilitating gene.

In another embodiment, there is provided a method for treating cancer comprising contacting a cancerous cell with an agent first identified as having gene modulating activity using any of the assay methods disclosed according to the invention and in an amount effective to reduce the cancerous activity of said cell. In a preferred embodiment, the cancerous cell is contacted in vivo. In other preferred embodiments, said reduction in cancerous activity is a decrease in the rate of proliferation of said cancerous cell, or said reduction in cancerous activity is the death of said cancerous cell.

The present invention further relates to a method for treating cancer comprising contacting a cancerous cell with an agent having activity against an expression product encoded by a gene corresponding to a polynucleotide comprising a nucleotide sequence as identified in Table 1 (SEQ ID NO: 1-923) where the product is a polypeptide, most preferably one comprising an amino acid sequence as identified in Table 1 (SEQ ID NO: 806-854). In a preferred embodiment, said cancerous cell is contacted in vivo. In another preferred embodiment, the agent is an antibody.

As noted, the genes useful in the assay methods include genes mapping within chromosomal regions of interest and genes as identified in Table 1 (SEQ ID NO: 1-923), or a gene that encodes the same RNA, such as the same messenger RNA, whose corresponding cDNA is one of the sequences as identified in Table 1 (SEQ ID NO: 1-923). The genes useful in the methods of the invention further include genes encoding RNAs whose corresponding cDNA is at least 90% identical to a sequence as identified in Table 1 (SEQ ID NO: 1-923), preferably at least about 95% identical to such a sequence, more preferably at least about 98% identical to such sequence and most preferably one comprising that sequence are specifically contemplated by all of the methods of the present invention.

In addition, sequences encoding the same proteins (SEQ ID NO: 806-854) as any of these sequences, regardless of the percent identity of such sequences, are also specifically contemplated by the invention.

The sequences disclosed herein may be genomic in nature and thus represent the sequence of an actual gene, such as a human gene, or may be a cDNA sequence derived from a messenger RNA (mRNA) and thus represent contiguous exonic sequences derived from a corresponding genomic sequence or they may be wholly synthetic in origin for purposes of testing. As described in the Example, the expression of these cancer-related genes is determined from the relative expression levels of the RNA complement of a cancerous cell relative to a normal (i.e., non-cancerous) cell. Because of the processing that may take place in transforming the initial RNA transcript into the final mRNA, the sequences disclosed herein may represent less than the full genomic sequence. They may also represent sequences derived from ribosomal and transfer RNAs. Consequently, the genes present in the cell (and representing the genomic sequences) and the sequences disclosed herein, which are mostly cDNA sequences, may be identical or may be such that the cDNAs contain less than the full genomic sequence. Such genes and cDNA sequences are still considered corresponding sequences because they both encode similar RNA sequences. Thus, by way of non-limiting example only, a gene that encodes an RNA transcript, which is then processed into a shorter mRNA, is deemed to encode both such RNAs and therefore encodes an RNA complementary to (using the usual Watson-Crick complementarity rules), or that would otherwise be encoded by, a cDNA (for example, a sequence as disclosed herein). Thus, the sequences disclosed herein correspond to genes contained in the cancerous or normal cells used to determine relative levels of expression because they represent the same sequences or are complementary to RNAs encoded by these genes. Such genes also include different alleles and splice variants that may occur in the cells used in the methods of the invention.

The genes of the invention “correspond to” a polynucleotide having a sequence as identified in Table 1 (SEQ ID NO: 1-923) if the gene encodes an RNA (processed or unprocessed, including naturally occurring splice variants and alleles) that is at least 90% identical, preferably at least 95% identical, most preferably at least 98% identical to, and especially identical to, an RNA that would be encoded by, or be complementary to, such as by hybridization with, a polynucleotide having the indicated sequence. In addition, genes including sequences at least 90% identical to a sequence as identified in Table 1 (SEQ ID NO: 1-923), preferably at least about 95% identical to such a sequence, more preferably at least about 98% identical to such sequence and most preferably comprising such sequence are specifically contemplated by all of the methods of the present invention as being genes that correspond to these sequences. In addition, sequences encoding the same proteins as any of these sequences, regardless of the percent identity of such sequences, are also specifically contemplated by any of the methods of the present invention that rely on any or all of said sequences, regardless of how they are otherwise described or limited. Thus, any such sequences are available for use in carrying out any of the methods disclosed according to the invention. Such sequences also include any open reading frames, as defined herein, present within any of the sequences as identified in Table 1 (SEQ ID NO: 1-805 and 855-923).

Further in accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100[1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. When used in relation to a polynucleotide, such terms refer to the products produced by treatment of said polynucleotides with any of the common endonucleases, or any stretch of polynucleotides that could be synthetically synthesized.

As used herein, the term “DNA segment” or “DNA sequence” refers to a DNA polymer, in the form of a separate fragment or as a component of a larger DNA construct, which has been derived from DNA, and may include both single stranded and duplex sequences. Such segments are provided in the form of an open reading frame uninterrupted by internal non-translated sequences, or introns, which are typically present in eukaryotic genes.

The term “coding region” refers to that portion of a gene which either naturally or normally codes for the expression product of that gene in its natural genomic environment, i.e., the region coding in vivo for the native expression product of the gene.

The term “nucleotide sequence” refers to a heteropolymer of deoxyribonucleotides. Generally, DNA segments encoding the proteins provided by this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

The term “expression product” means that polypeptide or protein that is the natural translation product of the gene and any nucleic acid sequence coding equivalents resulting from genetic code degeneracy and thus coding for the same amino acid(s).

The term “fragment,” when referring to a coding sequence, means a portion of DNA comprising less than the complete coding region whose expression product retains essentially the same biological function or activity as the expression product of the complete coding region.

The present invention also finds use as a means of diagnosing the presence of cancer in a patient, as where a sample of cancerous tissues or cells, or tissues or cells suspected of being cancerous. For such purposes, and in accordance with the disclosure elsewhere herein, such diagnosis is based on the detection of elevated expression or amplification, such as elevated copy number, of one or more of the genes identified according to the invention. Such elevated expression can be determined by any of the means described herein.

In one such embodiment, the elevated expression, as compared to normal cells and/or tissues of the same organ, is determined by measuring the relative rates of transcription of RNA, such as by production of corresponding cDNAs and then analyzing the resulting DNA using probes developed from the gene sequences as identified in Table 1. Thus, the levels of cDNA produced by use of reverse transcriptase with the full RNA complement of a cell suspected of being cancerous produces a corresponding amount of cDNA that can then be amplified using polymerase chain reaction, or some other means, such as rolling circle amplification, to determine the relative levels of resulting cDNA and, thereby, the relative levels of gene expression.

For RNA analysis, the latter may be isolated from samples in a variety of ways, including lysis and denaturation with a phenolic solution containing a chaotropic agent (e.g., triazol) followed by isopropanol precipitation, ethanol wash, and resuspension in aqueous solution; or lysis and denaturation followed by isolation on solid support, such as a Qiagen resin and reconstitution in aqueous solution; or lysis and denaturation in non-phenolic, aqueous solutions followed by enzymatic conversion of RNA to DNA template copies. Steady state RNA levels for a given type of cell or tissue may have to be ascertained prior to employment of the methods of the invention but such is well within the skill of those in the art and will not be further described in detail herein.

Alternatively, increased expression, such as increased copy number, may be determined for the genes present in a cancerous cell, or a cell suspected of being cancerous, by using the nucleotides sequences as identified in Table 1 as a means of generating probes for the DNAs present in the cells to be examined. Thus, the DNA of such cells may be extracted and probed using the sequences disclosed herein for the presence in the genomes of such cells of increased amounts of one or more of the genes of the invention. For example, where a cancer-related, or cancer-linked, gene as disclosed herein is found to be present in multiple copies within the genome of a cell, even where it may not be actively being over-expressed at the time of such determination, this may be indicative of at least a disposition toward developing cancer at a subsequent time.

In accordance with the foregoing, the presence of such multiple copies of a gene, or genes, as disclosed herein may be determined using northern or southern blotting and employing the sequences as identified in Table 1 to develop probes for this purpose. Such probes may be composed of DNA or RNA and may advantageously be comprised of a contiguous stretch of nucleotide residues matching, or complementary to, a sequence as identified in Table 1. Such probes will most usefully comprise a contiguous stretch of at least 15, preferably at least 30, more preferably at least 50, most preferably at least 80, and especially at least 100, even 200 residues, derived from one or more of the sequences as identified in Table 1. Thus, where a single probe binds multiple times to the genome of a sample of cells that are cancerous, or are suspected of being cancerous, or predisposed to become cancerous, whereas binding of the same probe to a similar amount of DNA derived from the genome of otherwise non-cancerous cells of the same organ or tissue results in observably less binding, this is indicative of the presence of multiple copies of a gene comprising, or corresponding to, the sequence as identified in Table 1 from which the probe sequenced was derived.

Increased expression may also be determined using agents that selectively bind to, and thereby detect, the presence of expression products of the genes disclosed herein. For example, an antibody, possibly a suitably labeled antibody, such as where the antibody is bound to a fluorescent or radiolabel, may be generated against one of the polypeptides comprising a sequence as identified in Table 1 (serial number 1-229 (breast), 230-440 (colon), 441-656 (lung) and 657-805 (prostate); for polypeptide SEQ ID NOs, see Table 1, serial number 806-923 (transcript or protein)), and said antibody will then react with, binding either selectively or specifically, to a polypeptide encoded by one of the genes that corresponds to a sequence disclosed herein. Such antibody binding, especially relative extent of such binding in samples derived from suspected cancerous, as opposed to otherwise non-cancerous, cells and tissues, can then be used as a measure of the extent of expression, or over-expression, of the cancer-related genes identified herein. Thus, the genes identified herein as being over-expressed in cancerous cells and tissues may be over-expressed due to increased copy number, or due to over-transcription, such as where the over-expression is due to over-production of a transcription factor that activates the gene and leads to repeated binding of RNA polymerase, thereby generating large than normal amounts of RNA transcripts, which are subsequently translated into polypeptides, such as the polypeptides comprising amino acid sequences as identified in table 1)SEQ ID NO: 1-923). Such analysis provides an additional means of ascertaining the expression of the genes identified according to the invention and thereby determining the presence of a cancerous state in a sample derived from a patient to be tested, of the predisposition to develop cancer at a subsequent time in said patient.

In employing the methods of the invention, it should be borne in mind that gene expression indicative of a cancerous state need not be characteristic of every cell found to be cancerous. Thus, the methods disclosed herein are useful for detecting the presence of a cancerous condition within a tissue where less than all cells exhibit the complete pattern of over-expression. For example, a set of selected genes, comprising sequences homologous under stringent conditions, or at least 90%, preferably 95%, identical to at least one of the sequences as identified in Table 1, may be found, using appropriate probes, either DNA or RNA, to be present in as little as 60% of cells derived from a sample of tumorous, or malignant, tissue while being absent from as much as 60% of cells derived from corresponding non-cancerous, or otherwise normal, tissue (and thus being present in as much as 40% of such normal tissue cells). In a preferred embodiment, such gene pattern is found to be present in at least 70% of cells drawn from a cancerous tissue and absent from at least 70% of a corresponding normal, non-cancerous, tissue sample. In an especially preferred embodiment, such gene pattern is found to be present in at least 80% of cells drawn from a cancerous tissue and absent from at least 80% of a corresponding normal, non-cancerous, tissue sample. In a most preferred embodiment, such gene pattern is found to be present in at least 90% of cells drawn from a cancerous tissue and absent from at least 90% of a corresponding normal, non-cancerous, tissue sample. In an additional embodiment, such gene pattern is found to be present in at least 100% of cells drawn from a cancerous tissue and absent from at least 100% of a corresponding normal, non-cancerous, tissue sample, although the latter embodiment may represent a rare occurrence.

In an additional aspect, the present invention relates to a method for determining a cancer initiating or facilitating gene comprising contacting a cell expressing a test gene (i.e., a gene whose status as a cancer initiating or facilitating gene is to be determined) with an agent that decreases the expression of a gene that encodes an RNA at least 90%, preferably 95%, identical to an RNA encoded by (i.e., a gene corresponding to) a polynucleotide comprising, or having, a sequence selected from the group consisting as identified in Table 1 and detecting a decrease in expression of said test gene compared to when said agent is not present, thereby identifying said test gene as being a cancer initiating or facilitating gene. Such genes may, of course, be oncogenes and said decrease in expression may be due to a decrease in copy number of said gene in said cell or a cell derived from said cell, such as where copy number is reduced in the cells formed by replication of such cells.

Thus, some or all of the genes disclosed herein as corresponding to as identified in Table 1 are found to play a direct role in the initiation or progression of cancer or even other diseases and disease processes. Because changes in expression of these genes (up-regulation) are linked to the disease state (i.e. cancer), the change in expression may contribute to the initiation or progression of the disease. For example, if a gene that is up-regulated is an oncogene such a gene provides for a means of screening for small molecule therapeutics beyond screens based upon expression output alone. For example, genes that display up-regulation in cancer and whose elevated expression contributes to initiation or progression of disease represent targets in screens for small molecules that inhibit or block their function. Examples include, but are not be limited to, kinase inhibition, cellular proliferation, substrate analogs that block the active site of protein targets, etc.

It should be noted that there are a variety of different contexts in which genes have been evaluated as being involved in the cancerous process. Thus, some genes may be oncogenes and encode proteins that are directly involved in the cancerous process and thereby promote the occurrence of cancer in an animal. Other genes may simply be involved either directly or indirectly in the cancerous process or condition and may serve in an ancillary capacity with respect to the cancerous state. All such types of genes are deemed with those to be determined in accordance with the invention as disclosed herein. Thus, the gene determined by said method of the invention may be an oncogene, or the gene determined by said method may be a cancer facilitating gene, the latter including a gene that directly or indirectly affects the cancerous process, either in the promotion of a cancerous condition or in facilitating the progress of cancerous growth or otherwise modulating the growth of cancer cells, either in vivo or ex vivo. Such genes may work indirectly where their expression alters the activity of some other gene or gene expression product that is itself directly involved in initiating or facilitating the progress of a cancerous condition. For example, a gene that encodes a polypeptide, either wild or mutant in type, which polypeptide acts to suppress of tumor suppressor gene, or its expression product, will thereby act indirectly to promote tumor growth.

In accordance with the foregoing, the method of the present invention includes cancer modulating agents that are themselves either polypeptides, or small chemical entities, that affect the cancerous process, including initiation, suppression or facilitation of tumor growth, either in vivo or ex vivo. Such agents may also be antibodies that react with one or more of the polypeptides as identified in Table 1 ((SEQ ID NO: 806-923 (transcript or protein)).

In keeping with the disclosure herein, the present invention also relates to a method for treating cancer comprising contacting a cancerous cell with an agent having activity against an expression product encoded by a gene mapping within regions of chromosomal interest or, alternatively, a gene corresponding to a polynucleotide that comprises a nucleotide sequence as identified in Table 1, such as where such expression product is one the polypeptides as identified in Table 1.

The method of the present invention includes embodiments of the above-recited method wherein said cancer cell is contacted in vivo as well as ex vivo, preferably wherein said agent comprises a portion, or is part of an overall molecular structure, having affinity for said expression product. In one such embodiment, said portion having affinity for said expression product is an antibody.

In one embodiment of the present invention, a chemical agent, such as a protein or other polypeptide, is joined to an agent, such as an antibody, having affinity for an expression product of a cancerous cell, such as a polypeptide or protein encoded by a gene related to the cancerous process, especially a gene sequence corresponding to one of the cDNA sequences as identified in Table 1. In a specific embodiment, said expression product acts as a therapeutic target for the affinity portion of said anticancer agent and where, after binding of the affinity portion of such agent to the expression product, the anti-cancer portion of said agent acts against said expression product so as to neutralize its effects in initiating, facilitating or promoting tumor formation and/or growth. In a separate embodiment of the present invention, binding of the agent to said expression product may, without more, have the effect of deterring cancer promotion, facilitation or growth, especially where the presence of said expression product is related, either intimately or only in an ancillary manner, to the development and growth of a tumor. Thus, where the presence of said expression product is essential to tumor initiation and/or growth, binding of said agent to said expression product will have the effect of negating said tumor promoting activity. In one such embodiment, said agent is an apoptosis-inducing agent that induces cell suicide, thereby killing the cancer cell and halting tumor growth.

Many cancers contain chromosomal rearrangements, which typically represent translocations, amplifications, or deletions of specific regions of genomic DNA. A recurrent chromosomal rearrangement that is associated with a specific stage and type of cancer always affects a gene (or possibly genes) that play a direct and critical role in the initiation or progression of the disease. Many of the known oncogenes or tumor suppressor genes that play direct roles in cancer have either been initially identified based upon their positional cloning from a recurrent chromosomal rearrangement or have been demonstrated to fall within a rearrangement subsequent to their cloning by other methods. In all cases, such genes display amplification at both the level of DNA copy number and at the level of transcriptional expression at the mRNA level.

The present method also relates to a method for determining functionally related genes comprising contacting one or more gene sequences corresponding to the cDNAs as identified in Table 1 with an agent that modulates expression of more than one gene in such group and thereby determining a subset of genes of said group.

In accordance with the present invention, said functionally related genes are genes modulating the same metabolic pathway or said genes are genes encoding functionally related polypeptides. In one such embodiment, said genes are genes whose expression is modulated by the same transcriptional activator or enhancer sequence, especially where said transcriptional activator or enhancer increases, or otherwise modulates, the activity of a gene corresponding to a cDNA as identified in Table 1.

The present invention also relates to a process that comprises a method for producing a product comprising identifying an agent according to one of the disclosed methods for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient. information to identify the compounds having a particular modulating activity (for example, the code number with the corresponding results). This transmission of information from user 1 to user 2 is specifically contemplated by the present invention.

In accordance with the foregoing, the present invention relates to a method for producing test data with respect to the anti-neoplastic activity of a compound comprising:

(a) contacting a compound with a cell that expresses at least one gene corresponding to a polynucleotide comprising a nucleotide sequence of serial number 1-229 (breast), 230-440 (colon), 441-656 (lung) and 657-805 (prostate) of Table 1 or encoding a polypeptide or transcript of SEQ ID NO:

806-923 and under conditions promoting expression of said gene;

(b) detecting a change in expression of said gene compared to expression when said compound is not present; and

(c) producing test data with respect to the gene modulating activity of said compound based on a change in the expression of the determined gene, or genes, whose expression is otherwise elevated in a non-cancerous cell over that in a cancerous cell and a decrease in the expression of the determined gene, or genes whose expression is otherwise increased in a cancerous cell over that in a non-cancerous cell indicating anti-neoplastic activity.

In another embodiment, the present invention provides a method for monitoring the progress of a cancer treatment, such as where the methods of the invention permit a determination that a given course of cancer therapy is or is not proving effective because of an increased or decreased expression of a gene, or genes, disclosed herein. For example, where there is an increased copy number of one or more of the genes as identified in Table 1 (SEQ ID NO: 1-805), monitoring of such genes can predict success or failure of a course of therapy, such as chemotherapy, or predict the likelihood of a relapse based on elevated activity or expression of one or more of these genes following such course of therapy.

In accordance with the foregoing, the present invention contemplates a method for determining the progress of a treatment for cancer in a patient afflicted with cancer, following commencement of a cancer treatment on said patient, comprising:

(a) determining in said patient a change in expression of one or more genes corresponding to a polynucleotide comprising a nucleotide sequence of serial number 1-229 (breast), 230-440 (colon), 441-656 (lung) and 657-805 (prostate) of Table 1 or encoding a polypeptide or transcript of serial number 806-923 of table 1) which include any of SEQ ID NO: 1-923) and under conditions promoting expression of said one or more genes; and

(b) detecting a change in expression of said gene compared to expression of said one or more determined genes prior to commencement of said cancer treatment;

thereby determining the progress of said treatment.

In a preferred embodiment, the detected change in expression is a decrease in expression. In another preferred embodiment, the cancer treatment is treatment with a chemotherapeutic agent, especially an agent that modulates, preferably decreases, expression of a gene identified herein, such as where said agent was first identified as having anti-neoplastic activity using a method of the invention. Thus, in accordance with this aspect of the present invention, a patient, or even a research animal, such as a mouse, rat, rabbit or primate, afflicted with cancer, including a cancer induced for research purposes, is introduced to a cancer treatment regimen, such as administration of an anti-cancer agent, including one first identified as having anti-neoplastic activity by one or more of the screening methods disclosed herein. The progress and success or failure of such treatment is subsequently ascertained by determining the subsequent expression of one or more, preferably at least 3, or 5, or 10, of the genes identified herein, or that encodes a transcript or polypeptide disclosed herein (see Table 1) following said treatment. In a preferred embodiment, a treatment that reduces said expression is deemed advantageous and may then be the basis for continuing said treatment. The methods of the invention thereby provide a means of continually monitoring the success of the treatment and evaluating both the need, and desirability, of continuing said treatment. In addition, more than one said treatment may be administered simultaneously without diminishing the value of the methods of the invention in determining the overall success of such combined treatment. Thus, more than one said anti-neoplastic agent may be administered to the same patient and overall effectiveness ascertained by the recited methods.

In accordance with the foregoing, the present invention also contemplates a method for determining the likelihood of survival of a patient afflicted with cancer, following commencement of a cancer treatment on said patient, comprising:

(a) determining in said patient a change in expression of one or more genes corresponding to a polynucleotide comprising a nucleotide sequence of serial number 1-229 (breast), 230-440 (colon), 441-656 (lung) and 657-805 (prostate) of Table 1 or encoding a polypeptide or transcript of serial number 806-923 of Table 1 and under conditions promoting expression of said one or more genes; and

(b) detecting a change in expression of said gene compared to expression of said one or more determined genes prior to commencement of said cancer treatment;

thereby determining the likelihood of survival of said treatment.

In a preferred embodiment, the detected change in expression is a decrease in expression and said determined gene, or genes, may include 2, 3, 5, 10 or more of the genes described herein. Thus, the methods of the invention may be utilized as a means for compiling cancer survival statistics following one or more, possibly combined, treatments for cancer as in keeping with the other methods disclosed herein.

The genes identified herein also offer themselves as pharmacodynamic markers (or as pharmacogenetic and/or surrogate markers), such as for patient profiling prior to clinical trials and/or targeted therapies, including combination treatments, resulting from the identification of these genes as valid gene targets for chemotherapy based on the screening procedures of the invention. In one embodiment thereof, the likelihood of success of a cancer treatment with a selected chemotherapeutic agent may be based on the fact that such agent has been determined to have expression modulating activity with one or more genes identified herein, especially where said genes have been identified as showing elevated expression levels in samples from a prospective patient afflicted with cancer. Methods described elsewhere herein for determining cancerous status of a cell may find ready use in such evaluations.

It should be cautioned that, in carrying out the procedures of the present invention as disclosed herein, any reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting example. In applying the disclosure of the example, it should be kept clearly in mind that other and different embodiments of the methods disclosed according to the present invention will no doubt suggest themselves to those of skill in the relevant art.

### EXAMPLE

Cancerous cells that over-express one or more of the genes selected from those that correspond to genes as identified in Table 1 (serial number 1-229 (breast), 230-440 (colon), 441-656 (lung) and 657-805 (prostate); serial number 806-923 (transcript or protein), or SEQ ID NO: 1-805 and 855-923) are grown to a density of 10⁵ cells/cm2 in Leibovitz's L-15 medium supplemented with 2 mM L-glutamine (90%) and 10% fetal bovine serum. The cells are collected after treatment with 0.25% trypsin, 0.02% EDTA at 37° C. for 2 to 5 minutes. The trypsinized cells are then diluted with 30 ml growth medium and plated at a density of 50,000 cells per well in a 96 well plate (200 μl/well). The following day, cells are treated with either compound buffer alone, or compound buffer containing a chemical agent to be tested, for 24 hours. The media is then removed, the cells lysed and the RNA recovered using the RNAeasy reagents and protocol obtained from Qiagen. RNA is quantitated and 10 ng of sample in 1 μl are added to 24 μl of Taqman reaction mix containing 1X PCR buffer, RNAsin, reverse transcriptase, nucleoside triphosphates, amplitaq gold, tween 20, glycerol, bovine serum albumin (BSA) and specific PCR primers and probes for a reference gene (18S RNA) and a test gene (Gene X). Reverse transcription is then carried out at 48° C. for 30 minutes. The sample is then applied to a Perlin Elmer 7700 sequence detector and heat denatured for 10 minutes at 95° C. Amplification is performed through 40 cycles using 15 seconds annealing at 60° C. followed by a 60 second extension at 72° C. and 30 second denaturation at 95° C. Data files are then captured and the data analyzed with the appropriate baseline windows and thresholds.

The quantitative difference between the target and reference genes is then calculated and a relative expression value determined for all of the samples used. This procedure is then repeated for each of the target genes in a given signature, or characteristic, set and the relative expression ratios for each pair of genes is determined (i.e., a ratio of expression is determined for each target gene versus each of the other genes for which expression is measured, where each gene's absolute expression is determined relative to the reference gene for each compound, or chemical agent, to be screened). The samples are then scored and ranked according to the degree of alteration of the expression profile in the treated samples relative to the control. The overall expression of the set of genes relative to the controls, as modulated by one chemical agent relative to another, is also ascertained. Chemical agents having the most effect on a given gene, or set of genes, are considered the most anti-neoplastic.

TABLE 1

| Serial | | | | | | | | | Protein/ |
|---|---|---|---|---|---|---|---|---|---|
| No. | SEQ ID | accession | tissue | p_m | chr | band | unigene | Description | Transcript |
| 1 | 3 | AK000490 | breast | primary | 1 | p31.2 | Hs.133260 | hypothetical protein FLJ20354 | |
| 2 | 10 | R33352 | breast | primary | 1 | p31.3 | NULL | unknown | |
| 3 | 13 | AI739473 | breast | primary | 1 | p32.3 | Hs.75616 | 24-dehydrocholesterol reductase | |
| 4 | 5 | U63743 | breast | primary | 1 | p34.1 | Hs.69360 | kinesin-like 6 (mitotic centromere-associated | |
| | | | | | | | | kinesin) | |
| 5 | 2 | U05340 | breast | primary | 1 | p34.2 | Hs.82906 | CDC20 cell division cycle 20 homolog (S. cerevisiae) | |
| 6 | 11 | AA203213 | breast | primary | 1 | p36.33 | Hs.833 | interferon-stimulated protein 15 kDa | |
| 7 | 12 | T16144 | breast | primary | 1 | q21.3 | NULL | unknown | |
| 8 | 1 | AI053741 | breast | primary | 1 | q22 | Hs.133294 | ESTs | |
| 9 | 14 | AB037776 | breast | primary | 1 | q23.1 | Hs.38002 | immunoglobulin superfamily member 9 | |
| 10 | 9 | AA830844 | breast | primary | 1 | q23.2 | Hs.127310 | kinase interacting with leukemia-associated | |
| | | | | | | | | gene (stathmin) | |
| 11 | 7 | AF326731 | breast | primary | 1 | q23.3 | Hs.234545 | cell division cycle associated 1 | |
| 12 | 4 | AB032931 | breast | primary | 1 | q32.1 | Hs.5199 | HSPC150 protein similar to ubiquitin- | |
| | | | | | | | | conjugating enzyme | |
| 13 | 8 | AI380204 | breast | primary | 1 | q32.1 | Hs.118064 | similar to rat nuclear ubiquitous casein kinase 2 | |
| 14 | 6 | U30872 | breast | primary | 1 | q32.3 | Hs.77204 | centromere protein F 350/400 ka (mitosin) | |
| 15 | 55 | U14518 | breast | primary | 2 | p23.3 | Hs.1594 | centromere protein A 17 kDa | |
| 16 | 54 | AI492879 | breast | primary | 2 | p25.1 | Hs.75319 | ribonucleotide reductase M2 polypeptide | |
| 17 | 56 | AL045632 | breast | primary | 2 | q33.1 | Hs.44269 | hypothetical protein FLJ25211 | |
| 18 | 74 | M86699 | breast | primary | 3 | p21.31 | Hs.169840 | TTK protein kinase | |
| 19 | 77 | AI962335 | breast | primary | 3 | p24.3 | Hs.196042 | ESTs | |
| 20 | 75 | AI867102 | breast | primary | 3 | p25.1 | Hs.56966 | KIAA0906 protein | |
| 21 | 71 | AI751438 | breast | primary | 3 | q12.3 | Hs.41271 | Homo sapiens mRNA full length insert cDNA | |
| | | | | | | | | clone EUROIMAGE 1913076 | |
| 22 | 72 | X57527 | breast | primary | 3 | q12.3 | Hs.114599 | collagen type VIII alpha 1 | |
| 23 | 76 | W02608 | breast | primary | 3 | q26.1 | Hs.36830 | ESTs Moderately similar to zinc finger protein | |
| | | | | | | | | 91 (HPF7 HTF10) [Homo sapiens] [H. sapiens] | |
| 24 | 73 | AI823992 | breast | primary | 3 | q26.32 | Hs.122579 | epithelial cell transforming sequence 2 | |
| | | | | | | | | oncogene | |
| 25 | 78 | AI087975 | breast | primary | 3 | q28 | Hs.195225 | ESTs | |
| 26 | 82 | AW001872 | breast | primary | 5 | p13.1 | Hs.58435 | FYN binding protein (FYB-120/130) | |
| 27 | 80 | BE407516 | breast | primary | 5 | q13.2 | Hs.23960 | cyclin B1 | |
| 28 | 81 | U70370 | breast | primary | 5 | q31.1 | Hs.84136 | paired-like homeodomain transcription factor 1 | |
| 29 | 79 | AI739117 | breast | primary | 5 | q31.2 | Hs.73625 | RAB6 interacting kinesin-like (rabkinesin6) | |
| 30 | 83 | D14678 | breast | primary | 6 | p21.32 | Hs.20830 | kinesin-like 2 | |
| 31 | 85 | M13436 | breast | primary | 7 | p14.1 | Hs.727 | inhibin beta A (activin A activin AB alpha | |
| | | | | | | | | polypeptide) | |
| 32 | 86 | AI343467 | breast | primary | 7 | p14.1 | Hs.28792 | Homo sapiens cDNA FLJ11041 fis clone | |
| | | | | | | | | PLACE1004405 | |
| 33 | 84 | AK023208 | breast | primary | 7 | p14.2 | Hs.62180 | anillin actin binding protein (scraps homolog | |
| | | | | | | | | Drosophila) | |
| 34 | 89 | AI285531 | breast | primary | 7 | p15.2 | Hs.106260 | sorting nexin 10 | |
| 35 | 87 | AI922323 | breast | primary | 7 | p21.1 | Hs.91011 | anterior gradient 2 homolog (Xenepus laevis) | |
| 36 | 88 | U61145 | breast | primary | 7 | q36.1 | Hs.77256 | enhancer of zeste homolog 2 (Drosophila) | |
| 37 | 99 | AA625199 | breast | primary | 8 | NULL | Hs.352415 | solute carrier family 39 (zinc transporter) | |
| | | | | | | | | member 4 | |
| 38 | 100 | AI949095 | breast | primary | 8 | NULL | Hs.67776 | Homo sapiens clone IMAGE: 5455669 mRNA | |
| | | | | | | | | partial cds | |
| 39 | 90 | AI932328 | breast | primary | 8 | p21.1 | Hs.104741 | T-LAK cell-originated protein kinase | |
| 40 | 91 | AA203476 | breast | primary | 8 | q13.2 | Hs.252587 | pituitary tumor-transforming 1 | |
| 41 | 92 | AW043713 | breast | primary | 8 | q13.3 | Hs.70823 | sulfatase FP | |
| 42 | 96 | BE974098 | breast | primary | 8 | q21.13 | Hs.2384 | tumor protein D52 | |
| 43 | 98 | AF091433 | breast | primary | 8 | q22.1 | Hs.30464 | cyclin E2 | |
| 44 | 95 | AA046853 | breast | primary | 8 | q24.12 | Hs.76550 | mal T-cell differentiation protein 2 | |
| 45 | 93 | AI925583 | breast | primary | 8 | q24.13 | Hs.222088 | hypothetical protein MGC5254 | |
| 46 | 97 | AF098865 | breast | primary | 8 | q24.13 | Hs.71465 | squalene epoxidase | |
| 47 | 94 | AA147884 | breast | primary | 8 | q24.22 | Hs.9812 | Homo sapiens cDNA FLJ14388 fis clone | |
| | | | | | | | | HEMBA1002716 | |
| 48 | 103 | AW007586 | breast | primary | 9 | q34.11 | Hs.133122 | zinc finger DHHC domain containing 12 | |
| 49 | 101 | W25552 | breast | primary | 9 | q34.3 | Hs.212613 | hypothetical protein FLJ36779 | |
| 50 | 102 | AI811865 | breast | primary | 9 | q34.3 | Hs.274152 | EST | |
| 51 | 17 | AF067656 | breast | primary | 10 | q21.1 | Hs.42650 | ZW10 interactor | |
| 52 | 16 | AL524035 | breast | primary | 10 | q21.2 | Hs.334562 | cell division cycle 2 G1 to S and G2 to M | |
| 53 | 15 | AI674163 | breast | primary | 10 | q23.33 | Hs.14559 | hypothetical protein FLJ10540 | |
| 54 | 21 | AB018293 | breast | primary | 11 | p15.3 | Hs.314434 | KIAA0750 gene product | |
| 55 | 18 | AL079372 | breast | primary | 11 | q13.1 | Hs.23044 | similar to RIKEN cDNA 2610036L13 | |
| 56 | 22 | D60944 | breast | primary | 11 | q13.4 | Hs.84700 | serologically defined colon cancer antigen 28 | |
| 57 | 19 | X14850 | breast | primary | 11 | q23.3 | Hs.147097 | H2A histone family member X | |
| 58 | 20 | AA704137 | breast | primary | 11 | q23.3 | Hs.125359 | Thy-1 cell surface antigen | |
| 59 | 23 | U74612 | breast | primary | 12 | p13.33 | Hs.239 | forkhead box M1 | |
| 60 | 24 | U82984 | breast | primary | 12 | q13.12 | Hs.23900 | Rac GTPase activating protein 1 | |
| 61 | 25 | AI291142 | breast | primary | 13 | q33.3 | Hs.183874 | cullin 4A | |
| 62 | 26 | L25876 | breast | primary | 14 | q22.1 | Hs.84113 | cyclin-dependent kinase inhibitor 3 (CDK2- | |
| | | | | | | | | associated dual specificity phosphatase) | |
| 63 | 27 | AL080146 | breast | primary | 15 | q21.3 | Hs.194698 | cyclin B2 | |
| 64 | 28 | D14657 | breast | primary | 15 | q22.2 | Hs.81892 | KIAA0101 gene product | |
| 65 | 29 | AA195614 | breast | primary | 15 | q25.3 | Hs.344037 | protein regulator of cytokinesis 1 | |
| 66 | 31 | AW003626 | breast | primary | 16 | NULL | Hs.159154 | tubulin beta 4 | |
| 67 | 32 | BC003186 | breast | primary | 16 | NULL | Hs.108196 | HSPC037 protein | |
| 68 | 30 | AI819340 | breast | primary | 16 | p13.3 | Hs.13561 | hypothetical protein MGC4692 | |
| 69 | 34 | W92110 | breast | primary | 16 | p13.3 | Hs.279623 | selenoprotein × 1 | |
| 70 | 35 | AI953838 | breast | primary | 16 | p13.3 | Hs.124015 | hypothetical protein MGC2605 | |
| 71 | 36 | AL520675 | breast | primary | 16 | p13.3 | Hs.351474 | hypothetical protein FLJ30002 | |
| 72 | 37 | BE965311 | breast | primary | 16 | p13.3 | Hs.124915 | hypothetical protein MGC2601 | |
| 73 | 38 | AI701742 | breast | primary | 16 | p13.3 | Hs.290943 | Homo sapiens similar to possible G-protein | |
| | | | | | | | | receptor clone MGC: 21993 IMAGE: 4398317 | |
| | | | | | | | | mRNA complete cds | |
| 74 | 33 | AA904482 | breast | primary | 16 | q12.2 | Hs.368078 | ESTs | |
| 75 | 42 | AI683036 | breast | primary | 17 | NULL | Hs.314169 | KIAA1618 protein | |
| 76 | 44 | U81800 | breast | primary | 17 | NULL | Hs.85838 | solute carrier family 16 (monocarboxylic acid | |
| | | | | | | | | transporters) member 3 | |
| 77 | 45 | BE328850 | breast | primary | 17 | q11.2 | Hs.348504 | hypothetical protein BC014072 | |
| 78 | 39 | AW003286 | breast | primary | 17 | q21.31 | Hs.370428 | ESTs Moderately similar to TP2A_HUMAN | |
| | | | | | | | | DNA topoisomerase II alpha isozyme | |
| | | | | | | | | [H. sapiens] | |
| 79 | 41 | AL561834 | breast | primary | 17 | q21.31 | Hs.156346 | topoisomerase (DNA) II alpha 170 kDa | |
| 80 | 48 | L47276 | breast | primary | 17 | q21.31 | NULL | unknown | |
| 81 | 49 | BC001038 | breast | primary | 17 | q22 | Hs.307036 | Homo sapiens Similar to epsin 3 clone | |
| | | | | | | | | MGC: 1006 IMAGE: 3505495 mRNA complete | |
| | | | | | | | | cds | |
| 82 | 40 | AA424160 | breast | primary | 17 | q23.2 | Hs.165909 | ESTs Weakly similar to hypothetical protein | |
| | | | | | | | | FLJ20489 [Homo sapiens] [H. sapiens] | |
| 83 | 51 | BF029215 | breast | primary | 17 | q23.2 | Hs.103512 | Homo sapiens cDNA FLJ36569 fis clone | |
| | | | | | | | | TRACH2010824 highly similar to | |
| | | | | | | | | Ribonucleoprotein | |
| 84 | 43 | AI675178 | breast | primary | 17 | q24.2 | Hs.90207 | hypothetical protein MGC11138 | |
| 85 | 50 | U28386 | breast | primary | 17 | q24.3 | Hs.159557 | karyopherin alpha 2 (RAG cohort 1 importin | |
| | | | | | | | | alpha 1) | |
| 86 | 46 | AA635844 | breast | primary | 17 | q25.3 | Hs.109706 | hematological and neurological expressed 1 | |
| 87 | 47 | K02581 | breast | primary | 17 | q25.3 | Hs.105097 | thymidine kinase 1 soluble | |
| 88 | 52 | AF017790 | breast | primary | 18 | p11.32 | Hs.58169 | highly expressed in cancer rich in leucine | |
| | | | | | | | | heptad repeats | |
| 89 | 53 | AA719022 | breast | primary | 19 | q13.43 | Hs.288549 | ubiquitin UBF-fl | |
| 90 | 65 | D80008 | breast | primary | 20 | p11.21 | Hs.36232 | KIAA0186 gene product | |
| 91 | 63 | AI990405 | breast | primary | 20 | p11.23 | Hs.194691 | retinoic acid induced 3 | |
| 92 | 57 | AA534688 | breast | primary | 20 | q11.1 | Hs.9329 | chromosome 20 open reading frame 1 | |
| 93 | 66 | AW003586 | breast | primary | 20 | q11.22 | Hs.274411 | SCAN domain containing 1 | |
| 94 | 59 | U73379 | breast | primary | 20 | q13.12 | Hs.93002 | ubiquitin-conjugating enzyme E2C | |
| 95 | 62 | AI990026 | breast | primary | 20 | q13.12 | Hs.286 | ribosomal protein L4 | |
| 96 | 67 | AA207074 | breast | primary | 20 | q13.13 | Hs.56237 | breast carcinoma amplified sequence 4 | |
| 97 | 60 | AF041260 | breast | primary | 20 | q13.2 | Hs.129057 | breast carcinoma amplified sequence 1 | |
| 98 | 61 | AF011468 | breast | primary | 20 | q13.31 | Hs.250822 | serine/threonine kinase 6 | |
| 99 | 58 | AA535819 | breast | primary | 20 | q13.32 | Hs.83883 | transmembrane prostate androgen induced | |
| | | | | | | | | RNA | |
| 100 | 64 | X70940 | breast | primary | 20 | q13.33 | Hs.2642 | eukaryotic translation elongation factor 1 alpha 2 | |
| 101 | 69 | Y15915 | breast | primary | 22 | q13.1 | Hs.172928 | collagen type I alpha 1 | |
| 102 | 70 | AL035081 | breast | primary | 22 | q13.1 | Hs.250696 | KDEL (Lys-Asp-Glu-Leu) endoplasmic | |
| | | | | | | | | reticulum protein retention receptor 3 | |
| 103 | 68 | AI381686 | breast | primary | 22 | q13.2 | Hs.208912 | hypothetical protein MGC861 | |
| 104 | 106 | AK000490 | breast | metastatic | 1 | p31.2 | Hs.133260 | hypothetical protein FLJ20354 | |
| 105 | 113 | R33352 | breast | metastatic | 1 | p31.3 | NULL | unknown | |
| 106 | 116 | AI739473 | breast | metastatic | 1 | p32.3 | Hs.75616 | 24-dehydrocholesterol reductase | |
| 107 | 108 | U63743 | breast | metastatic | 1 | p34.1 | Hs.69360 | kinesin-like 6 (mitotic centromere-associated | |
| | | | | | | | | kinesin) | |
| 108 | 105 | U05340 | breast | metastatic | 1 | p34.2 | Hs.82906 | CDC20 cell division cycle 20 homolog (S. cerevisiae) | |
| 109 | 119 | AI992172 | breast | metastatic | 1 | p36.13 | Hs.83551 | microfibrillar-associated protein 2 | |
| 110 | 114 | AA203213 | breast | metastatic | 1 | p36.33 | Hs.833 | interferon-stimulated protein 15 kDa | |
| 111 | 115 | T16144 | breast | metastatic | 1 | q21.3 | NULL | unknown | |
| 112 | 104 | AI053741 | breast | metastatic | 1 | q22 | Hs.133294 | ESTs | |
| 113 | 118 | AB037776 | breast | metastatic | 1 | q23.1 | Hs.38002 | immunoglobulin superfamily member 9 | |
| 114 | 112 | AA830844 | breast | metastatic | 1 | q23.2 | Hs.127310 | kinase interacting with leukemia-associated | |
| | | | | | | | | gene (stathmin) | |
| 115 | 120 | R62346 | breast | metastatic | 1 | q23.2 | NULL | unknown | |
| 116 | 110 | AF326731 | breast | metastatic | 1 | q23.3 | Hs.234545 | cell division cycle associated 1 | |
| 117 | 117 | AI983896 | breast | metastatic | 1 | q23.3 | Hs.191187 | ESTs | |
| 118 | 121 | AI798144 | breast | metastatic | 1 | q25.2 | Hs.209609 | ESTs | |
| 119 | 107 | AI990409 | breast | metastatic | 1 | q32.1 | Hs.5199 | HSPC150 protein similar to ubiquitin- | |
| | | | | | | | | conjugating enzyme | |
| 120 | 111 | AI380204 | breast | metastatic | 1 | q32.1 | Hs.118064 | similar to rat nuclear ubiquitous casein kinase 2 | |
| 121 | 109 | U30872 | breast | metastatic | 1 | q32.3 | Hs.77204 | centromere protein F 350/400 ka (mitosin) | |
| 122 | 169 | U14518 | breast | metastatic | 2 | p23.3 | Hs.1594 | centromere protein A 17 kDa | |
| 123 | 168 | AI492879 | breast | metastatic | 2 | p25.1 | Hs.75319 | ribonucleotide reductase M2 polypeptide | |
| 124 | 170 | AL045632 | breast | metastatic | 2 | q33.1 | Hs.44269 | hypothetical protein FLJ25211 | |
| 125 | 171 | N21131 | breast | metastatic | 2 | q37.3 | Hs.42949 | hairy and enhancer of split 6 (Drosophila) | |
| 126 | 191 | M86699 | breast | metastatic | 3 | p21.31 | Hs.169840 | TTK protein kinase | |
| 127 | 197 | AA663786 | breast | metastatic | 3 | p21.31 | NULL | unknown | |
| 128 | 194 | AI962335 | breast | metastatic | 3 | p24.3 | Hs.196042 | ESTs | |
| 129 | 195 | AB020713 | breast | metastatic | 3 | p25.1 | Hs.56966 | KIAA0906 protein | |
| 130 | 188 | AI557210 | breast | metastatic | 3 | q12.3 | Hs.41271 | Homo sapiens mRNA full length insert cDNA | |
| | | | | | | | | clone EUROIMAGE 1913076 | |
| 131 | 189 | X57527 | breast | metastatic | 3 | q12.3 | Hs.114599 | collagen type VIII alpha 1 | |
| 132 | 192 | W02608 | breast | metastatic | 3 | q26.1 | Hs.36830 | ESTs Moderately similar to zinc finger protein | |
| | | | | | | | | 91 (HPF7 HTF10) [Homo sapiens] [H. sapiens] | |
| 133 | 193 | AI760298 | breast | metastatic | 3 | q26.31 | Hs.128773 | ESTs | |
| 134 | 190 | AI823992 | breast | metastatic | 3 | q26.32 | Hs.122579 | epithelial cell transforming sequence 2 | |
| | | | | | | | | oncogene | |
| 135 | 196 | AI087975 | breast | metastatic | 3 | q28 | Hs.195225 | ESTs | |
| 136 | 201 | AW001872 | breast | metastatic | 5 | p13.1 | Hs.58435 | FYN binding protein (FYB-120/130) | |
| 137 | 199 | N90191 | breast | metastatic | 5 | q13.2 | Hs.23960 | cyclin B1 | |
| 138 | 200 | U70370 | breast | metastatic | 5 | q31.1 | Hs.84136 | paired-like homeodomain transcription factor 1 | |
| 139 | 198 | AI739117 | breast | metastatic | 5 | q31.2 | Hs.73625 | RAB6 interacting kinesin-like (rabkinesin6) | |
| 140 | 202 | D14678 | breast | metastatic | 6 | p21.32 | Hs.20830 | kinesin-like 2 | |
| 141 | 204 | M13436 | breast | metastatic | 7 | p14.1 | Hs.727 | inhibin beta A (activin A activin AB alpha | |
| | | | | | | | | polypeptide) | |
| 142 | 205 | AA059458 | breast | metastatic | 7 | p14.1 | Hs.28792 | Homo sapiens cDNA FLJ11041 fis clone | |
| | | | | | | | | PLACE1004405 | |
| 143 | 203 | AK023208 | breast | metastatic | 7 | p14.2 | Hs.62180 | anillin actin binding protein (scraps homolog | |
| | | | | | | | | Drosophila) | |
| 144 | 211 | AI742239 | breast | metastatic | 7 | p15.1 | Hs.91109 | Homo sapiens Similar to RIKEN cDNA | |
| | | | | | | | | E130201N16 gene clone IMAGE: 3845782 | |
| | | | | | | | | mRNA | |
| 145 | 208 | AI285531 | breast | metastatic | 7 | p15.2 | Hs.106260 | sorting nexin 10 | |
| 146 | 206 | AI922323 | breast | metastatic | 7 | p21.1 | Hs.91011 | anterior gradient 2 homolog (Xenepus laevis) | |
| 147 | 209 | AI961907 | breast | metastatic | 7 | q21.3 | Hs.179573 | collagen type I alpha 2 | |
| 148 | 210 | L37127 | breast | metastatic | 7 | q22.1 | Hs.80475 | polymerase (RNA) II (DNA directed) | |
| | | | | | | | | polypeptide J 13.3 kDa | |
| 149 | 207 | U61145 | breast | metastatic | 7 | q36.1 | Hs.77256 | enhancer of zeste homolog 2 (Drosophila) | |
| 150 | 220 | AA625199 | breast | metastatic | 8 | NULL | Hs.352415 | solute carrier family 39 (zinc transporter) | |
| | | | | | | | | member 4 | |
| 151 | 223 | AI949095 | breast | metastatic | 8 | NULL | Hs.67776 | Homo sapiens clone IMAGE: 5455669 mRNA | |
| | | | | | | | | partial cds | |
| 152 | 224 | W22510 | breast | metastatic | 8 | NULL | Hs.346950 | cellular retinoic acid binding protein 1 | |
| 153 | 225 | AA292431 | breast | metastatic | 8 | NULL | Hs.92679 | kinesin family member C2-like | |
| 154 | 226 | AI917311 | breast | metastatic | 8 | NULL | Hs.149152 | rhophilin 1 | |
| 155 | 212 | AI932328 | breast | metastatic | 8 | p21.1 | Hs.104741 | T-LAK cell-originated protein kinase | |
| 156 | 213 | AA203476 | breast | metastatic | 8 | q13.2 | Hs.252587 | pituitary tumor-transforming 1 | |
| 157 | 215 | BE500977 | breast | metastatic | 8 | q13.3 | Hs.70823 | sulfatase FP | |
| 158 | 217 | BE974098 | breast | metastatic | 8 | q21.13 | Hs.2384 | tumor protein D52 | |
| 159 | 219 | AF091433 | breast | metastatic | 8 | q22.1 | Hs.30464 | cyclin E2 | |
| 160 | 222 | AA610522 | breast | metastatic | 8 | q24.11 | Hs.162697 | ESTs | |
| 161 | 216 | AA046853 | breast | metastatic | 8 | q24.12 | Hs.76550 | mal T-cell differentiation protein 2 | |
| 162 | 218 | AI656807 | breast | metastatic | 8 | q24.13 | Hs.222088 | hypothetical protein MGC5254 | |
| 163 | 221 | D78130 | breast | metastatic | 8 | q24.13 | Hs.71465 | squalene epoxidase | |
| 164 | 214 | AA147884 | breast | metastatic | 8 | q24.22 | Hs.9812 | Homo sapiens cDNA FLJ14388 fis clone | |
| | | | | | | | | HEMBA1002716 | |
| 165 | 229 | AW007586 | breast | metastatic | 9 | q34.11 | Hs.133122 | zinc finger DHHC domain containing 12 | |
| 166 | 227 | W25552 | breast | metastatic | 9 | q34.3 | Hs.212613 | hypothetical protein FLJ36779 | |
| 167 | 228 | AI811865 | breast | metastatic | 9 | q34.3 | Hs.274152 | EST | |
| 168 | 124 | AF067656 | breast | metastatic | 10 | q21.1 | Hs.42650 | ZW10 interactor | |
| 169 | 123 | D88357 | breast | metastatic | 10 | q21.2 | Hs.334562 | cell division cycle 2 G1 to S and G2 to M | |
| 170 | 122 | AI674163 | breast | metastatic | 10 | q23.33 | Hs.14559 | hypothetical protein FLJ10540 | |
| 171 | 125 | U37426 | breast | metastatic | 10 | q23.33 | Hs.8878 | kinesin-like 1 | |
| 172 | 131 | AA705015 | breast | metastatic | 11 | p15.1 | Hs.185918 | Homo sapiens cDNA FLJ32525 fis clone | |
| | | | | | | | | SMINT2000060 | |
| 173 | 129 | AB018293 | breast | metastatic | 11 | p15.3 | Hs.314434 | KIAA0750 gene product | |
| 174 | 126 | AL079372 | breast | metastatic | 11 | q13.1 | Hs.23044 | similar to RIKEN cDNA 2610036L13 | |
| 175 | 130 | AF151810 | breast | metastatic | 11 | q13.4 | Hs.84700 | serologically defined colon cancer antigen 28 | |
| 176 | 127 | X14850 | breast | metastatic | 11 | q23.3 | Hs.147097 | H2A histone family member X | |
| 177 | 128 | AA704137 | breast | metastatic | 11 | q23.3 | Hs.125359 | Thy-1 cell surface antigen | |
| 178 | 132 | U74612 | breast | metastatic | 12 | p13.33 | Hs.239 | forkhead box M1 | |
| 179 | 133 | U82984 | breast | metastatic | 12 | q13.12 | Hs.23900 | Rac GTPase activating protein 1 | |
| 180 | 134 | R61322 | breast | metastatic | 12 | q24.31 | Hs.204166 | Human clone 295 5cM region surrounding | |
| | | | | | | | | hepatocyte nuclear factor-1a/MODY3 mRNA | |
| 181 | 135 | AI291142 | breast | metastatic | 13 | q33.3 | Hs.183874 | cullin 4A | |
| 182 | 136 | L25876 | breast | metastatic | 14 | q22.1 | Hs.84113 | cyclin-dependent kinase inhibitor 3 (CDK2- | |
| | | | | | | | | associated dual specificity phosphatase) | |
| 183 | 137 | AL080146 | breast | metastatic | 15 | q21.3 | Hs.194698 | cyclin B2 | |
| 184 | 138 | D14657 | breast | metastatic | 15 | q22.2 | Hs.81892 | KIAA0101 gene product | |
| 185 | 139 | AA195614 | breast | metastatic | 15 | q25.3 | Hs.344037 | protein regulator of cytokinesis 1 | |
| 186 | 141 | AW003626 | breast | metastatic | 16 | NULL | Hs.159154 | tubulin beta 4 | |
| 187 | 142 | BC003186 | breast | metastatic | 16 | NULL | Hs.108196 | HSPC037 protein | |
| 188 | 149 | AI766311 | breast | metastatic | 16 | p12.3 | Hs.289047 | Homo sapiens cDNA FLJ14059 fis clone | |
| | | | | | | | | HEMBB1000573 | |
| 189 | 151 | AI344053 | breast | metastatic | 16 | p12.3 | Hs.115838 | ESTs Highly similar to hypothetical protein | |
| | | | | | | | | FLJ13593 [Homo sapiens] [H. sapiens] | |
| 190 | 140 | AI819340 | breast | metastatic | 16 | p13.3 | Hs.13561 | hypothetical protein MGC4692 | |
| 191 | 144 | W92110 | breast | metastatic | 16 | p13.3 | Hs.279623 | selenoprotein × 1 | |
| 192 | 145 | AI953838 | breast | metastatic | 16 | p13.3 | Hs.124015 | hypothetical protein MGC2605 | |
| 193 | 146 | AL520675 | breast | metastatic | 16 | p13.3 | Hs.351474 | hypothetical protein FLJ30002 | |
| 194 | 147 | BE965311 | breast | metastatic | 16 | p13.3 | Hs.124915 | hypothetical protein MGC2601 | |
| 195 | 148 | AI701742 | breast | metastatic | 16 | p13.3 | Hs.290943 | Homo sapiens similar to possible G-protein | |
| | | | | | | | | receptor clone MGC: 21993 IMAGE: 4398317 | |
| | | | | | | | | mRNA complete cds | |
| 196 | 150 | AI655799 | breast | metastatic | 16 | p13.3 | Hs.197114 | serine/arginine repetitive matrix 2 | |
| 197 | 143 | AA904482 | breast | metastatic | 16 | q12.2 | Hs.368078 | ESTs | |
| 198 | 154 | AI683036 | breast | metastatic | 17 | NULL | Hs.314169 | KIAA1618 protein | |
| 199 | 156 | U81800 | breast | metastatic | 17 | NULL | Hs.85838 | solute carrier family 16 (monocarboxylic acid | |
| | | | | | | | | transporters) member 3 | |
| 200 | 157 | BE328850 | breast | metastatic | 17 | q11.2 | Hs.348504 | hypothetical protein BC014072 | |
| 201 | 152 | AW003286 | breast | metastatic | 17 | q21.31 | Hs.370428 | ESTs Moderately similar to TP2A_HUMAN | |
| | | | | | | | | DNA topoisomerase II alpha isozyme | |
| | | | | | | | | [H. sapiens] | |
| 202 | 158 | AI375913 | breast | metastatic | 17 | q21.31 | Hs.156346 | topoisomerase (DNA) II alpha 170 kDa | |
| 203 | 161 | L47276 | breast | metastatic | 17 | q21.31 | NULL | unknown | |
| 204 | 162 | BC001038 | breast | metastatic | 17 | q22 | Hs.307036 | Homo sapiens Similar to epsin 3 clone | |
| | | | | | | | | MGC: 1006 IMAGE: 3505495 mRNA complete | |
| | | | | | | | | cds | |
| 205 | 153 | AA424160 | breast | metastatic | 17 | q23.2 | Hs.165909 | ESTs Weakly similar to hypothetical protein | |
| | | | | | | | | FLJ20489 [Homo sapiens] [H. sapiens] | |
| 206 | 164 | BF029215 | breast | metastatic | 17 | q23.2 | Hs.103512 | Homo sapiens cDNA FLJ36569 fis clone | |
| | | | | | | | | TRACH2010824 highly similar to | |
| | | | | | | | | Ribonucleoprotein | |
| 207 | 155 | AI675178 | breast | metastatic | 17 | q24.2 | Hs.90207 | hypothetical protein MGC11138 | |
| 208 | 163 | U28386 | breast | metastatic | 17 | q24.3 | Hs.159557 | karyopherin alpha 2 (RAG cohort 1 importin | |
| | | | | | | | | alpha 1) | |
| 209 | 165 | N42752 | breast | metastatic | 17 | q24.3 | Hs.42645 | ESTs | |
| 210 | 159 | K02581 | breast | metastatic | 17 | q25.3 | Hs.105097 | thymidine kinase 1 soluble | |
| 211 | 160 | AI525822 | breast | metastatic | 17 | q25.3 | Hs.109706 | hematological and neurological expressed 1 | |
| 212 | 166 | AF017790 | breast | metastatic | 18 | p11.32 | Hs.58169 | highly expressed in cancer rich in leucine | |
| | | | | | | | | heptad repeats | |
| 213 | 167 | AA719022 | breast | metastatic | 19 | q13.43 | Hs.288549 | ubiquitin UBF-fl | |
| 214 | 180 | D80008 | breast | metastatic | 20 | p11.21 | Hs.36232 | KIAA0186 gene product | |
| 215 | 178 | AI990405 | breast | metastatic | 20 | p11.23 | Hs.194691 | retinoic acid induced 3 | |
| 216 | 177 | AF098158 | breast | metastatic | 20 | q11.1 | Hs.9329 | chromosome 20 open reading frame 1 | |
| 217 | 181 | AW003586 | breast | metastatic | 20 | q11.22 | Hs.274411 | SCAN domain containing 1 | |
| 218 | 173 | U73379 | breast | metastatic | 20 | q13.12 | Hs.93002 | ubiquitin-conjugating enzyme E2C | |
| 219 | 176 | AI990026 | breast | metastatic | 20 | q13.12 | Hs.286 | ribosomal protein L4 | |
| 220 | 182 | AA207074 | breast | metastatic | 20 | q13.13 | Hs.56237 | breast carcinoma amplified sequence 4 | |
| 221 | 174 | AF041260 | breast | metastatic | 20 | q13.2 | Hs.129057 | breast carcinoma amplified sequence 1 | |
| 222 | 183 | AI638036 | breast | metastatic | 20 | q13.2 | Hs.189095 | sal-like 4 (Drosophila) | |
| 223 | 175 | AF011468 | breast | metastatic | 20 | q13.31 | Hs.250822 | serine/threonine kinase 6 | |
| 224 | 172 | AA535819 | breast | metastatic | 20 | q13.32 | Hs.83883 | transmembrane prostate androgen induced | |
| | | | | | | | | RNA | |
| 225 | 179 | X70940 | breast | metastatic | 20 | q13.33 | Hs.2642 | eukaryotic translation elongation factor 1 alpha 2 | |
| 226 | 184 | AI872267 | breast | metastatic | 20 | q13.33 | Hs.224895 | ESTs | |
| 227 | 186 | Y15915 | breast | metastatic | 22 | q13.1 | Hs.172928 | collagen type I alpha 1 | |
| 228 | 187 | AL035081 | breast | metastatic | 22 | q13.1 | Hs.250696 | KDEL (Lys-Asp-Glu-Leu) endoplasmic | |
| | | | | | | | | reticulum protein retention receptor 3 | |
| 229 | 185 | AI961206 | breast | metastatic | 22 | q13.2 | Hs.208912 | hypothetical protein MGC861 | |
| 230 | 241 | AK000490 | colon | primary | 1 | p31.2 | Hs.133260 | hypothetical protein FLJ20354 | |
| 231 | 235 | R33352 | colon | primary | 1 | p31.3 | NULL | unknown | |
| 232 | 233 | AI739473 | colon | primary | 1 | p32.3 | Hs.75616 | 24-dehydrocholesterol reductase | |
| 233 | 239 | U63743 | colon | primary | 1 | p34.1 | Hs.69360 | kinesin-like 6 (mitotic centromere-associated | |
| | | | | | | | | kinesin) | |
| 234 | 243 | U05340 | colon | primary | 1 | p34.2 | Hs.82906 | CDC20 cell division cycle 20 homolog (S. cerevisiae) | |
| 235 | 242 | AI990026 | colon | primary | 1 | p35.3 | Hs.286 | ribosomal protein L4 | |
| 236 | 234 | T16144 | colon | primary | 1 | q21.3 | NULL | unknown | |
| 237 | 232 | AW271106 | colon | primary | 1 | q22 | Hs.133294 | ESTs | |
| 238 | 230 | AB037776 | colon | primary | 1 | q23.1 | Hs.38002 | immunoglobulin superfamily member 9 | |
| 239 | 236 | AA830844 | colon | primary | 1 | q23.2 | Hs.127310 | kinase interacting with leukemia-associated | |
| | | | | | | | | gene (stathmin) | |
| 240 | 231 | AA383718 | colon | primary | 1 | q23.3 | Hs.234545 | cell division cycle associated 1 | |
| 241 | 237 | AI380204 | colon | primary | 1 | q32.1 | Hs.118064 | similar to rat nuclear ubiquitous casein kinase 2 | |
| 242 | 240 | AI990409 | colon | primary | 1 | q32.1 | Hs.5199 | HSPC150 protein similar to ubiquitin- | |
| | | | | | | | | conjugating enzyme | |
| 243 | 238 | U30872 | colon | primary | 1 | q32.3 | Hs.77204 | centromere protein F 350/400 ka (mitosin) | |
| 244 | 284 | U14518 | colon | primary | 2 | p23.3 | Hs.1594 | centromere protein A 17 kDa | |
| 245 | 285 | BE966236 | colon | primary | 2 | p25.1 | Hs.75319 | ribonucleotide reductase M2 polypeptide | |
| 246 | 283 | AL045632 | colon | primary | 2 | q33.1 | Hs.44269 | hypothetical protein FLJ25211 | |
| 247 | 302 | M86699 | colon | primary | 3 | p21.31 | Hs.169840 | TTK protein kinase | |
| 248 | 301 | AI962335 | colon | primary | 3 | p24.3 | Hs.196042 | ESTs | |
| 249 | 300 | AB020713 | colon | primary | 3 | p25.1 | Hs.56966 | KIAA0906 protein | |
| 250 | 304 | X57527 | colon | primary | 3 | q12.3 | Hs.114599 | collagen type VIII alpha 1 | |
| 251 | 305 | AI557210 | colon | primary | 3 | q12.3 | Hs.41271 | Homo sapiens mRNA full length insert cDNA | |
| | | | | | | | | clone EUROIMAGE 1913076 | |
| 252 | 303 | AI823992 | colon | primary | 3 | q26.32 | Hs.122579 | epithelial cell transforming sequence 2 | |
| | | | | | | | | oncogene | |
| 253 | 299 | AI087975 | colon | primary | 3 | q28 | Hs.195225 | ESTs | |
| 254 | 307 | AW001872 | colon | primary | 5 | p13.1 | Hs.58435 | FYN binding protein (FYB-120/130) | |
| 255 | 306 | M25753 | colon | primary | 5 | q13.2 | Hs.23960 | cyclin B1 | |
| 256 | 308 | U70370 | colon | primary | 5 | q31.1 | Hs.84136 | paired-like homeodomain transcription factor 1 | |
| 257 | 309 | AI739117 | colon | primary | 5 | q31.2 | Hs.73625 | RAB6 interacting kinesin-like (rabkinesin6) | |
| 258 | 310 | D14678 | colon | primary | 6 | p21.32 | Hs.20830 | kinesin-like 2 | |
| 259 | 313 | AA059458 | colon | primary | 7 | p14.1 | Hs.28792 | Homo sapiens cDNA FLJ11041 fis clone | |
| | | | | | | | | PLACE1004405 | |
| 260 | 315 | M13436 | colon | primary | 7 | p14.1 | Hs.727 | inhibin beta A (activin A activin AB alpha | |
| | | | | | | | | polypeptide) | |
| 261 | 314 | AI341261 | colon | primary | 7 | p14.2 | Hs.62180 | anillin actin binding protein (scraps homolog | |
| | | | | | | | | Drosophila) | |
| 262 | 312 | AI922323 | colon | primary | 7 | p21.1 | Hs.91011 | anterior gradient 2 homolog (Xenepus laevis) | |
| 263 | 311 | U61145 | colon | primary | 7 | q36.1 | Hs.77256 | enhancer of zeste homolog 2 (Drosophila) | |
| 264 | 316 | AI949095 | colon | primary | 8 | NULL | Hs.67776 | Homo sapiens clone IMAGE: 5455669 mRNA | |
| | | | | | | | | partial cds | |
| 265 | 318 | AA625199 | colon | primary | 8 | NULL | Hs.352415 | solute carrier family 39 (zinc transporter) | |
| | | | | | | | | member 4 | |
| 266 | 325 | AI932328 | colon | primary | 8 | p21.1 | Hs.104741 | T-LAK cell-originated protein kinase | |
| 267 | 324 | AA203476 | colon | primary | 8 | q13.2 | Hs.252587 | pituitary tumor-transforming 1 | |
| 268 | 323 | AW043713 | colon | primary | 8 | q13.3 | Hs.70823 | sulfatase FP | |
| 269 | 319 | AF091433 | colon | primary | 8 | q22.1 | Hs.30464 | cyclin E2 | |
| 270 | 321 | AL117612 | colon | primary | 8 | q24.12 | Hs.76550 | mal T-cell differentiation protein 2 | |
| 271 | 317 | D78130 | colon | primary | 8 | q24.13 | Hs.71465 | squalene epoxidase | |
| 272 | 320 | AI656807 | colon | primary | 8 | q24.13 | Hs.222088 | hypothetical protein MGC5254 | |
| 273 | 322 | AA147884 | colon | primary | 8 | q24.22 | Hs.9812 | Homo sapiens cDNA FLJ14388 fis clone | |
| | | | | | | | | HEMBA1002716 | |
| 274 | 326 | AW007586 | colon | primary | 9 | q34.11 | Hs.133122 | zinc finger DHHC domain containing 12 | |
| 275 | 327 | AI811865 | colon | primary | 9 | q34.3 | Hs.274152 | EST | |
| 276 | 328 | W25552 | colon | primary | 9 | q34.3 | Hs.212613 | hypothetical protein FLJ36779 | |
| 277 | 244 | AF067656 | colon | primary | 10 | q21.1 | Hs.42650 | ZW10 interactor | |
| 278 | 245 | AL524035 | colon | primary | 10 | q21.2 | Hs.334562 | cell division cycle 2 G1 to S and G2 to M | |
| 279 | 246 | AI674163 | colon | primary | 10 | q23.33 | Hs.14559 | hypothetical protein FLJ10540 | |
| 280 | 248 | AB018293 | colon | primary | 11 | p15.3 | Hs.314434 | KIAA0750 gene product | |
| 281 | 251 | AL079372 | colon | primary | 11 | q13.1 | Hs.23044 | similar to RIKEN cDNA 2610036L13 | |
| 282 | 247 | D60944 | colon | primary | 11 | q13.4 | Hs.84700 | serologically defined colon cancer antigen 28 | |
| 283 | 249 | AA704137 | colon | primary | 11 | q23.3 | Hs.125359 | Thy-1 cell surface antigen | |
| 284 | 250 | X14850 | colon | primary | 11 | q23.3 | Hs.147097 | H2A histone family member X | |
| 285 | 253 | U74612 | colon | primary | 12 | p13.33 | Hs.239 | forkhead box M1 | |
| 286 | 252 | U82984 | colon | primary | 12 | q13.12 | Hs.23900 | Rac GTPase activating protein 1 | |
| 287 | 254 | AI291142 | colon | primary | 13 | q33.3 | Hs.183874 | cullin 4A | |
| 288 | 255 | L25876 | colon | primary | 14 | q22.1 | Hs.84113 | cyclin-dependent kinase inhibitor 3 (CDK2- | |
| | | | | | | | | associated dual specificity phosphatase) | |
| 289 | 258 | AL080146 | colon | primary | 15 | q21.3 | Hs.194698 | cyclin B2 | |
| 290 | 257 | D14657 | colon | primary | 15 | q22.2 | Hs.81892 | KIAA0101 gene product | |
| 291 | 256 | AA195614 | colon | primary | 15 | q25.3 | Hs.344037 | protein regulator of cytokinesis 1 | |
| 292 | 265 | BC003186 | colon | primary | 16 | NULL | Hs.108196 | HSPC037 protein | |
| 293 | 266 | AW003626 | colon | primary | 16 | NULL | Hs.159154 | tubulin beta 4 | |
| 294 | 259 | AI701742 | colon | primary | 16 | p13.3 | Hs.290943 | Homo sapiens similar to possible G-protein | |
| | | | | | | | | receptor clone MGC: 21993 IMAGE: 4398317 | |
| | | | | | | | | mRNA complete cds | |
| 295 | 260 | BE965311 | colon | primary | 16 | p13.3 | Hs.124915 | hypothetical protein MGC2601 | |
| 296 | 261 | AL520675 | colon | primary | 16 | p13.3 | Hs.351474 | hypothetical protein FLJ30002 | |
| 297 | 262 | AI953838 | colon | primary | 16 | p13.3 | Hs.124015 | hypothetical protein MGC2605 | |
| 298 | 263 | W92110 | colon | primary | 16 | p13.3 | Hs.279623 | selenoprotein × 1 | |
| 299 | 267 | AI819340 | colon | primary | 16 | p13.3 | Hs.13561 | hypothetical protein MGC4692 | |
| 300 | 264 | AA904482 | colon | primary | 16 | q12.2 | Hs.368078 | ESTs | |
| 301 | 276 | U81800 | colon | primary | 17 | NULL | Hs.85838 | solute carrier family 16 (monocarboxylic acid | |
| | | | | | | | | transporters) member 3 | |
| 302 | 278 | AI683036 | colon | primary | 17 | NULL | Hs.314169 | KIAA1618 protein | |
| 303 | 275 | BE328850 | colon | primary | 17 | q11.2 | Hs.348504 | hypothetical protein BC014072 | |
| 304 | 272 | L47276 | colon | primary | 17 | q21.31 | NULL | unknown | |
| 305 | 274 | AI375913 | colon | primary | 17 | q21.31 | Hs.156346 | topoisomerase (DNA) II alpha 170 kDa | |
| 306 | 280 | AW003286 | colon | primary | 17 | q21.31 | Hs.370428 | ESTs Moderately similar to TP2A_HUMAN | |
| | | | | | | | | DNA topoisomerase II alpha isozyme | |
| | | | | | | | | [H. sapiens] | |
| 307 | 271 | BC001038 | colon | primary | 17 | q22 | Hs.307036 | Homo sapiens Similar to epsin 3 clone | |
| | | | | | | | | MGC: 1006 IMAGE: 3505495 mRNA complete | |
| | | | | | | | | cds | |
| 308 | 269 | BF029215 | colon | primary | 17 | q23.2 | Hs.103512 | Homo sapiens cDNA FLJ36569 fis clone | |
| | | | | | | | | TRACH2010824 highly similar to | |
| | | | | | | | | Ribonucleoprotein | |
| 309 | 279 | BG165011 | colon | primary | 17 | q23.2 | Hs.165909 | ESTs Weakly similar to hypothetical protein | |
| | | | | | | | | FLJ20489 [Homo sapiens] [H. sapiens] | |
| 310 | 277 | AI675178 | colon | primary | 17 | q24.2 | Hs.90207 | hypothetical protein MGC11138 | |
| 311 | 270 | U28386 | colon | primary | 17 | q24.3 | Hs.159557 | karyopherin alpha 2 (RAG cohort 1 importin | |
| | | | | | | | | alpha 1) | |
| 312 | 268 | AI525822 | colon | primary | 17 | q25.3 | Hs.109706 | hematological and neurological expressed 1 | |
| 313 | 273 | K02581 | colon | primary | 17 | q25.3 | Hs.105097 | thymidine kinase 1 soluble | |
| 314 | 281 | AF017790 | colon | primary | 18 | p11.32 | Hs.58169 | highly expressed in cancer rich in leucine | |
| | | | | | | | | heptad repeats | |
| 315 | 282 | AA719022 | colon | primary | 19 | q13.43 | Hs.288549 | ubiquitin UBF-fl | |
| 316 | 288 | D80008 | colon | primary | 20 | p11.21 | Hs.36232 | KIAA0186 gene product | |
| 317 | 290 | AI990405 | colon | primary | 20 | p11.23 | Hs.194691 | retinoic acid induced 3 | |
| 318 | 291 | AF098158 | colon | primary | 20 | q11.1 | Hs.9329 | chromosome 20 open reading frame 1 | |
| 319 | 287 | AW003586 | colon | primary | 20 | q11.22 | Hs.274411 | SCAN domain containing 1 | |
| 320 | 294 | U73379 | colon | primary | 20 | q13.12 | Hs.93002 | ubiquitin-conjugating enzyme E2C | |
| 321 | 286 | AA207074 | colon | primary | 20 | q13.13 | Hs.56237 | breast carcinoma amplified sequence 4 | |
| 322 | 293 | AF041260 | colon | primary | 20 | q13.2 | Hs.129057 | breast carcinoma amplified sequence 1 | |
| 323 | 292 | AF011468 | colon | primary | 20 | q13.31 | Hs.250822 | serine/threonine kinase 6 | |
| 324 | 295 | AA535819 | colon | primary | 20 | q13.32 | Hs.83883 | transmembrane prostate androgen induced | |
| | | | | | | | | RNA | |
| 325 | 289 | X70940 | colon | primary | 20 | q13.33 | Hs.2642 | eukaryotic translation elongation factor 1 alpha 2 | |
| 326 | 296 | AL035081 | colon | primary | 22 | q13.1 | Hs.250696 | KDEL (Lys-Asp-Glu-Leu) endoplasmic | |
| | | | | | | | | reticulum protein retention receptor 3 | |
| 327 | 297 | Y15916 | colon | primary | 22 | q13.1 | Hs.172928 | collagen type I alpha 1 | |
| 328 | 298 | AI381686 | colon | primary | 22 | q13.2 | Hs.208912 | hypothetical protein MGC861 | |
| 329 | 420 | AK000490 | colon | metastatic | 1 | p31.2 | Hs.133260 | hypothetical protein FLJ20354 | |
| 330 | 354 | R33352 | colon | metastatic | 1 | p31.3 | NULL | unknown | |
| 331 | 351 | AI739473 | colon | metastatic | 1 | p32.3 | Hs.75616 | 24-dehydrocholesterol reductase | |
| 332 | 396 | U63743 | colon | metastatic | 1 | p34.1 | Hs.69360 | kinesin-like 6 (mitotic centromere-associated | |
| | | | | | | | | kinesin) | |
| 333 | 425 | U05340 | colon | metastatic | 1 | p34.2 | Hs.82906 | CDC20 cell division cycle 20 homolog (S. cerevisiae) | |
| 334 | 421 | AI990026 | colon | metastatic | 1 | p35.3 | Hs.286 | ribosomal protein L4 | |
| 335 | 352 | T16144 | colon | metastatic | 1 | q21.3 | NULL | unknown | |
| 336 | 346 | AW271106 | colon | metastatic | 1 | q22 | Hs.133294 | ESTs | |
| 337 | 340 | AB037776 | colon | metastatic | 1 | q23.1 | Hs.38002 | immunoglobulin superfamily member 9 | |
| 338 | 357 | AA830844 | colon | metastatic | 1 | q23.2 | Hs.127310 | kinase interacting with leukemia-associated | |
| | | | | | | | | gene (stathmin) | |
| 339 | 330 | AF326731 | colon | metastatic | 1 | q23.3 | Hs.234545 | cell division cycle associated 1 | |
| 340 | 341 | AI983896 | colon | metastatic | 1 | q23.3 | Hs.191187 | ESTs | |
| 341 | 380 | AF326731 | colon | metastatic | 1 | q23.3 | Hs.234545 | cell division cycle associated 1 | |
| 342 | 360 | AI380204 | colon | metastatic | 1 | q32.1 | Hs.118064 | similar to rat nuclear ubiquitous casein kinase 2 | |
| 343 | 403 | AI990409 | colon | metastatic | 1 | q32.1 | Hs.5199 | HSPC150 protein similar to ubiquitin- | |
| | | | | | | | | conjugating enzyme | |
| 344 | 382 | U30872 | colon | metastatic | 1 | q32.3 | Hs.77204 | centromere protein F 350/400 ka (mitosin) | |
| 345 | 400 | U14518 | colon | metastatic | 2 | p23.3 | Hs.1594 | centromere protein A 17 kDa | |
| 346 | 419 | BE966236 | colon | metastatic | 2 | p25.1 | Hs.75319 | ribonucleotide reductase M2 polypeptide | |
| 347 | 349 | AL045632 | colon | metastatic | 2 | q33.1 | Hs.44269 | hypothetical protein FLJ25211 | |
| 348 | 365 | M86699 | colon | metastatic | 3 | p21.31 | Hs.169840 | TTK protein kinase | |
| 349 | 334 | AI962335 | colon | metastatic | 3 | p24.3 | Hs.196042 | ESTs | |
| 350 | 333 | AB020713 | colon | metastatic | 3 | p25.1 | Hs.56966 | KIAA0906 protein | |
| 351 | 412 | X57527 | colon | metastatic | 3 | q12.3 | Hs.114599 | collagen type VIII alpha 1 | |
| 352 | 417 | AI557210 | colon | metastatic | 3 | q12.3 | Hs.41271 | Homo sapiens mRNA full length insert cDNA | |
| | | | | | | | | clone EUROIMAGE 1913076 | |
| 353 | 344 | W02608 | colon | metastatic | 3 | q26.1 | Hs.36830 | ESTs Moderately similar to zinc finger protein | |
| | | | | | | | | 91 (HPF7 HTF10) [Homo sapiens] [H. sapiens] | |
| 354 | 395 | AI823992 | colon | metastatic | 3 | q26.32 | Hs.122579 | epithelial cell transforming sequence 2 | |
| | | | | | | | | oncogene | |
| 355 | 332 | AI087975 | colon | metastatic | 3 | q28 | Hs.195225 | ESTs | |
| 356 | 409 | AW001872 | colon | metastatic | 5 | p13.1 | Hs.58435 | FYN binding protein (FYB-120/130) | |
| 357 | 408 | M25753 | colon | metastatic | 5 | q13.2 | Hs.23960 | cyclin B1 | |
| 358 | 410 | U70370 | colon | metastatic | 5 | q31.1 | Hs.84136 | paired-like homeodomain transcription factor 1 | |
| 359 | 434 | AI739117 | colon | metastatic | 5 | q31.2 | Hs.73625 | RAB6 interacting kinesin-like (rabkinesin6) | |
| 360 | 389 | D14678 | colon | metastatic | 6 | p21.32 | Hs.20830 | kinesin-like 2 | |
| 361 | 427 | AA059458 | colon | metastatic | 7 | p14.1 | Hs.28792 | Homo sapiens cDNA FLJ11041 fis clone | |
| | | | | | | | | PLACE1004405 | |
| 362 | 436 | AI343467 | colon | metastatic | 7 | p14.1 | Hs.28792 | Homo sapiens cDNA FLJ11041 fis clone | |
| | | | | | | | | PLACE1004405 | |
| 363 | 437 | M13436 | colon | metastatic | 7 | p14.1 | Hs.727 | inhibin beta A (activin A activin AB alpha | |
| | | | | | | | | polypeptide) | |
| 364 | 329 | AK023208 | colon | metastatic | 7 | p14.2 | Hs.62180 | anillin actin binding protein (scraps homolog | |
| | | | | | | | | Drosophila) | |
| 365 | 438 | AK023208 | colon | metastatic | 7 | p14.2 | Hs.62180 | anillin actin binding protein (scraps homolog | |
| | | | | | | | | Drosophila) | |
| 366 | 440 | AK023208 | colon | metastatic | 7 | p14.2 | Hs.62180 | anillin actin binding protein (scraps homolog | |
| | | | | | | | | Drosophila) | |
| 367 | 348 | AI285531 | colon | metastatic | 7 | p15.2 | Hs.106260 | sorting nexin 10 | |
| 368 | 392 | AI922323 | colon | metastatic | 7 | p21.1 | Hs.91011 | anterior gradient 2 homolog (Xenepus laevis) | |
| 369 | 339 | AI961907 | colon | metastatic | 7 | q21.3 | Hs.179573 | collagen type I alpha 2 | |
| 370 | 356 | U61145 | colon | metastatic | 7 | q36.1 | Hs.77256 | enhancer of zeste homolog 2 (Drosophila) | |
| 371 | 336 | AI949095 | colon | metastatic | 8 | NULL | Hs.67776 | Homo sapiens clone IMAGE: 5455669 mRNA | |
| | | | | | | | | partial cds | |
| 372 | 362 | AA625199 | colon | metastatic | 8 | NULL | Hs.352415 | solute carrier family 39 (zinc transporter) | |
| | | | | | | | | member 4 | |
| 373 | 439 | AI932328 | colon | metastatic | 8 | p21.1 | Hs.104741 | T-LAK cell-originated protein kinase | |
| 374 | 428 | AA203476 | colon | metastatic | 8 | q13.2 | Hs.252587 | pituitary tumor-transforming 1 | |
| 375 | 414 | AW043713 | colon | metastatic | 8 | q13.3 | Hs.70823 | sulfatase FP | |
| 376 | 345 | AA524023 | colon | metastatic | 8 | q21.13 | Hs.2384 | tumor protein D52 | |
| 377 | 363 | AF091433 | colon | metastatic | 8 | q22.1 | Hs.30464 | cyclin E2 | |
| 378 | 342 | AA610522 | colon | metastatic | 8 | q24.11 | Hs.162697 | ESTs | |
| 379 | 373 | AL117612 | colon | metastatic | 8 | q24.12 | Hs.76550 | mal T-cell differentiation protein 2 | |
| 380 | 347 | D78130 | colon | metastatic | 8 | q24.13 | Hs.71465 | squalene epoxidase | |
| 381 | 366 | AI656807 | colon | metastatic | 8 | q24.13 | Hs.222088 | hypothetical protein MGC5254 | |
| 382 | 388 | AA147884 | colon | metastatic | 8 | q24.22 | Hs.9812 | Homo sapiens cDNA FLJ14388 fis clone | |
| | | | | | | | | HEMBA1002716 | |
| 383 | 374 | AW007586 | colon | metastatic | 9 | q34.11 | Hs.133122 | zinc finger DHHC domain containing 12 | |
| 384 | 383 | AI811865 | colon | metastatic | 9 | q34.3 | Hs.274152 | EST | |
| 385 | 393 | W25552 | colon | metastatic | 9 | q34.3 | Hs.212613 | hypothetical protein FLJ36779 | |
| 386 | 355 | AF067656 | colon | metastatic | 10 | q21.1 | Hs.42650 | ZW10 interactor | |
| 387 | 368 | X05360 | colon | metastatic | 10 | q21.2 | Hs.334562 | cell division cycle 2 G1 to S and G2 to M | |
| 388 | 416 | AI674163 | colon | metastatic | 10 | q23.33 | Hs.14559 | hypothetical protein FLJ10540 | |
| 389 | 337 | AA705015 | colon | metastatic | 11 | p15.1 | Hs.185918 | Homo sapiens cDNA FLJ32525 fis clone | |
| | | | | | | | | SMINT2000060 | |
| 390 | 372 | AB018293 | colon | metastatic | 11 | p15.3 | Hs.314434 | KIAA0750 gene product | |
| 391 | 401 | AL079372 | colon | metastatic | 11 | q13.1 | Hs.23044 | similar to RIKEN cDNA 2610036L13 | |
| 392 | 350 | D60944 | colon | metastatic | 11 | q13.4 | Hs.84700 | serologically defined colon cancer antigen 28 | |
| 393 | 379 | AA704137 | colon | metastatic | 11 | q23.3 | Hs.125359 | Thy-1 cell surface antigen | |
| 394 | 381 | X14850 | colon | metastatic | 11 | q23.3 | Hs.147097 | H2A histone family member X | |
| 395 | 429 | U74612 | colon | metastatic | 12 | p13.33 | Hs.239 | forkhead box M1 | |
| 396 | 384 | U82984 | colon | metastatic | 12 | q13.12 | Hs.23900 | Rac GTPase activating protein 1 | |
| 397 | 343 | R61322 | colon | metastatic | 12 | q24.31 | Hs.204166 | Human clone 295 5cM region surrounding | |
| | | | | | | | | hepatocyte nuclear factor-1a/MODY3 mRNA | |
| 398 | 353 | AI291142 | colon | metastatic | 13 | q33.3 | Hs.183874 | cullin 4A | |
| 399 | 387 | L25876 | colon | metastatic | 14 | q22.1 | Hs.84113 | cyclin-dependent kinase inhibitor 3 (CDK2- | |
| | | | | | | | | associated dual specificity phosphatase) | |
| 400 | 423 | AL080146 | colon | metastatic | 15 | q21.3 | Hs.194698 | cyclin B2 | |
| 401 | 413 | D14657 | colon | metastatic | 15 | q22.2 | Hs.81892 | KIAA0101 gene product | |
| 402 | 406 | AA195614 | colon | metastatic | 15 | q25.3 | Hs.344037 | protein regulator of cytokinesis 1 | |
| 403 | 407 | BC003186 | colon | metastatic | 16 | NULL | Hs.108196 | HSPC037 protein | |
| 404 | 415 | AW003626 | colon | metastatic | 16 | NULL | Hs.159154 | tubulin beta 4 | |
| 405 | 358 | AI701742 | colon | metastatic | 16 | p13.3 | Hs.290943 | Homo sapiens similar to possible G-protein | |
| | | | | | | | | receptor clone MGC: 21993 IMAGE: 4398317 | |
| | | | | | | | | mRNA complete cds | |
| 406 | 361 | BE965311 | colon | metastatic | 16 | p13.3 | Hs.124915 | hypothetical protein MGC2601 | |
| 407 | 364 | AL520675 | colon | metastatic | 16 | p13.3 | Hs.351474 | hypothetical protein FLJ30002 | |
| 408 | 377 | AI953838 | colon | metastatic | 16 | p13.3 | Hs.124015 | hypothetical protein MGC2605 | |
| 409 | 385 | W92110 | colon | metastatic | 16 | p13.3 | Hs.279623 | selenoprotein × 1 | |
| 410 | 431 | AI819340 | colon | metastatic | 16 | p13.3 | Hs.13561 | hypothetical protein MGC4692 | |
| 411 | 405 | AA904482 | colon | metastatic | 16 | q12.2 | Hs.368078 | ESTs | |
| 412 | 391 | U81800 | colon | metastatic | 17 | NULL | Hs.85838 | solute carrier family 16 (monocarboxylic acid | |
| | | | | | | | | transporters) member 3 | |
| 413 | 411 | AI683036 | colon | metastatic | 17 | NULL | Hs.314169 | KIAA1618 protein | |
| 414 | 390 | BE328850 | colon | metastatic | 17 | q11.2 | Hs.348504 | hypothetical protein BC014072 | |
| 415 | 376 | L47276 | colon | metastatic | 17 | q21.31 | NULL | unknown | |
| 416 | 386 | AI375913 | colon | metastatic | 17 | q21.31 | Hs.156346 | topoisomerase (DNA) II alpha 170 kDa | |
| 417 | 432 | AW003286 | colon | metastatic | 17 | q21.31 | Hs.370428 | ESTs Moderately similar to TP2A_HUMAN | |
| | | | | | | | | DNA topoisomerase II alpha isozyme | |
| | | | | | | | | [H. sapiens] | |
| 418 | 375 | BC001038 | colon | metastatic | 17 | q22 | Hs.307036 | Homo sapiens Similar to epsin 3 clone | |
| | | | | | | | | MGC: 1006 IMAGE: 3505495 mRNA complete | |
| | | | | | | | | cds | |
| 419 | 359 | BF029215 | colon | metastatic | 17 | q23.2 | Hs.103512 | Homo sapiens cDNA FLJ36569 fis clone | |
| | | | | | | | | TRACH2010824 highly similar to | |
| | | | | | | | | Ribonucleoprotein | |
| 420 | 426 | BG165011 | colon | metastatic | 17 | q23.2 | Hs.165909 | ESTs Weakly similar to hypothetical protein | |
| | | | | | | | | FLJ20489 [Homo sapiens] [H. sapiens] | |
| 421 | 398 | AI675178 | colon | metastatic | 17 | q24.2 | Hs.90207 | hypothetical protein MGC11138 | |
| 422 | 369 | U28386 | colon | metastatic | 17 | q24.3 | Hs.159557 | karyopherin alpha 2 (RAG cohort 1 importin | |
| | | | | | | | | alpha 1) | |
| 423 | 335 | AI525822 | colon | metastatic | 17 | q25.3 | Hs.109706 | hematological and neurological expressed 1 | |
| 424 | 378 | K02581 | colon | metastatic | 17 | q25.3 | Hs.105097 | thymidine kinase 1 soluble | |
| 425 | 331 | AF017790 | colon | metastatic | 18 | p11.32 | Hs.58169 | highly expressed in cancer rich in leucine | |
| | | | | | | | | heptad repeats | |
| 426 | 404 | AF017790 | colon | metastatic | 18 | p11.32 | Hs.58169 | highly expressed in cancer rich in leucine | |
| | | | | | | | | heptad repeats | |
| 427 | 422 | AA719022 | colon | metastatic | 19 | q13.43 | Hs.288549 | ubiquitin UBF-fl | |
| 428 | 371 | D80008 | colon | metastatic | 20 | p11.21 | Hs.36232 | KIAA0186 gene product | |
| 429 | 399 | AI990405 | colon | metastatic | 20 | p11.23 | Hs.194691 | retinoic acid induced 3 | |
| 430 | 402 | AF098158 | colon | metastatic | 20 | q11.1 | Hs.9329 | chromosome 20 open reading frame 1 | |
| 431 | 370 | AW003586 | colon | metastatic | 20 | q11.22 | Hs.274411 | SCAN domain containing 1 | |
| 432 | 433 | U73379 | colon | metastatic | 20 | q13.12 | Hs.93002 | ubiquitin-conjugating enzyme E2C | |
| 433 | 338 | AA207074 | colon | metastatic | 20 | q13.13 | Hs.56237 | breast carcinoma amplified sequence 4 | |
| 434 | 430 | AF041260 | colon | metastatic | 20 | q13.2 | Hs.129057 | breast carcinoma amplified sequence 1 | |
| 435 | 424 | AF011468 | colon | metastatic | 20 | q13.31 | Hs.250822 | serine/threonine kinase 6 | |
| 436 | 435 | AA535819 | colon | metastatic | 20 | q13.32 | Hs.83883 | transmembrane prostate androgen induced | |
| | | | | | | | | RNA | |
| 437 | 394 | X70940 | colon | metastatic | 20 | q13.33 | Hs.2642 | eukaryotic translation elongation factor 1 alpha 2 | |
| 438 | 367 | AL035081 | colon | metastatic | 22 | q13.1 | Hs.250696 | KDEL (Lys-Asp-Glu-Leu) endoplasmic | |
| | | | | | | | | reticulum protein retention receptor 3 | |
| 439 | 397 | Y15916 | colon | metastatic | 22 | q13.1 | Hs.172928 | collagen type I alpha 1 | |
| 440 | 418 | AI381686 | colon | metastatic | 22 | q13.2 | Hs.208912 | hypothetical protein MGC861 | |
| 441 | 506 | AA905821 | lung | primary | 1 | p31.3 | Hs.145958 | ESTs | |
| 442 | 508 | AI056599 | lung | primary | 1 | p31.3 | Hs.120893 | ESTs | |
| 443 | 511 | AW070459 | lung | primary | 1 | p31.3 | Hs.259438 | ESTs | |
| 444 | 527 | AK022113 | lung | primary | 1 | p31.3 | Hs.301858 | Homo sapiens cDNA FLJ13017 fis clone | |
| | | | | | | | | NT2RP3000628 | |
| 445 | 528 | AU151151 | lung | primary | 1 | p31.3 | Hs.11493 | Homo sapiens cDNA FLJ13536 fis clone | |
| | | | | | | | | PLACE1006521 | |
| 446 | 547 | AB044807 | lung | primary | 1 | p31.3 | Hs.321197 | PDZ domain protein (Drosophila inaD-like) | |
| 447 | 485 | AA012917 | lung | primary | 1 | p32.1 | Hs.333541 | beta-amyloid binding protein precursor | |
| 448 | 498 | BF224444 | lung | primary | 1 | p32.1 | Hs.127274 | ESTs | |
| 449 | 526 | AU147177 | lung | primary | 1 | p32.1 | Hs.301237 | Homo sapiens cDNA FLJ12095 fis clone | |
| | | | | | | | | HEMBB1002610 | |
| 450 | 473 | AA926959 | lung | primary | 1 | q21.3 | Hs.77550 | p53-regulated DDA3 | |
| 451 | 443 | AI053741 | lung | primary | 1 | q22 | Hs.133294 | ESTs | |
| 452 | 448 | AI766666 | lung | primary | 1 | q22 | Hs.374850 | apolipoprotein A-I binding protein | |
| 453 | 469 | AI739071 | lung | primary | 1 | q22 | Hs.158515 | hypothetical protein MGC13038 | |
| 454 | 441 | AF326731 | lung | primary | 1 | q23.3 | Hs.234545 | cell division cycle associated 1 | |
| 455 | 446 | BC002906 | lung | primary | 1 | q23.3 | Hs.75939 | uridine monophosphate kinase | |
| 456 | 442 | AA182412 | lung | primary | 1 | q25.3 | Hs.32058 | chromosome 1 open reading frame 19 | |
| 457 | 482 | AA725362 | lung | primary | 2 | p11.1 | NULL | unknown | |
| 458 | 472 | AI990317 | lung | primary | 2 | p13.1 | Hs.154672 | methylene tetrahydrofolate dehydrogenase | |
| | | | | | | | | (NAD+ dependent) methenyltetrahydrofolate | |
| | | | | | | | | cyclohydrolase | |
| 459 | 464 | AI191897 | lung | primary | 2 | p16.2 | Hs.105223 | Homo sapiens Similar to RIKEN cDNA | |
| | | | | | | | | 2510006C20 gene clone MGC: 24001 | |
| | | | | | | | | IMAGE: 4050858 mRNA complete cds | |
| 460 | 474 | AI492879 | lung | primary | 2 | p25.1 | Hs.75319 | ribonucleotide reductase M2 polypeptide | |
| 461 | 481 | H24953 | lung | primary | 2 | q13 | NULL | unknown | |
| 462 | 451 | AA749314 | lung | primary | 2 | q31.1 | Hs.333893 | cell division cycle associated 7 | |
| 463 | 519 | C00851 | lung | primary | 5 | p13.2 | Hs.144264 | ESTs Weakly similar to hypothetical protein | |
| | | | | | | | | FLJ20837 [Homo sapiens] [H. sapiens] | |
| 464 | 458 | AA383208 | lung | primary | 5 | p15.1 | Hs.125249 | ESTs | |
| 465 | 548 | AA524353 | lung | primary | 6 | p21.2 | Hs.27693 | peptidylprolyl isomerase (cyclophilin)-like 1 | |
| 466 | 522 | AW005489 | lung | primary | 6 | p21.31 | Hs.139800 | high mobility group AT-hook 1 | |
| 467 | 538 | AI677701 | lung | primary | 6 | p22.3 | Hs.201619 | hypothetical protein FLJ30829 | |
| 468 | 551 | BG528420 | lung | primary | 6 | p22.3 | Hs.83484 | SRY (sex determining region Y)-box 4 | |
| 469 | 467 | AI439141 | lung | primary | 6 | p23 | Hs.261023 | hypothetical protein FLJ20958 | |
| 470 | 539 | AI279547 | lung | primary | 6 | p24.1 | Hs.8645 | hypothetical protein LOC51256 | |
| 471 | 540 | W27692 | lung | primary | 6 | p24.2 | Hs.273077 | hypothetical protein MGC1223 | |
| 472 | 495 | K03193 | lung | primary | 7 | p11.2 | Hs.77432 | epidermal growth factor receptor | |
| | | | | | | | | (erythroblastic leukemia viral (v-erb-b) | |
| | | | | | | | | oncogene homolog avian) | |
| 473 | 496 | AI806160 | lung | primary | 7 | p11.2 | Hs.127991 | ESTs | |
| 474 | 497 | AW138673 | lung | primary | 7 | p11.2 | Hs.252928 | ESTs | |
| 475 | 502 | H65306 | lung | primary | 7 | p11.2 | Hs.205559 | ESTs | |
| 476 | 509 | AW971863 | lung | primary | 7 | p11.2 | Hs.103351 | ESTs | |
| 477 | 536 | D60436 | lung | primary | 7 | p11.2 | Hs.335933 | Homo sapiens clone MGC: 33530 | |
| | | | | | | | | IMAGE: 4820705 mRNA complete cds | |
| 478 | 545 | AI363001 | lung | primary | 7 | p11.2 | Hs.134342 | LanC lantibiotic synthetase component C-like | |
| | | | | | | | | 2 (bacterial) | |
| 479 | 524 | AV700815 | lung | primary | 7 | p12.3 | Hs.180171 | Homo sapiens cDNA FLJ10417 fis clone | |
| | | | | | | | | NT2RP1000112 | |
| 480 | 486 | AA740186 | lung | primary | 7 | p13 | Hs.81029 | biliverdin reductase A | |
| 481 | 510 | AI252004 | lung | primary | 7 | p13 | Hs.284148 | ESTs | |
| 482 | 514 | AW452419 | lung | primary | 7 | p13 | Hs.296098 | ESTs | |
| 483 | 515 | AI418313 | lung | primary | 7 | p13 | Hs.152895 | ESTs | |
| 484 | 517 | AI191118 | lung | primary | 7 | p13 | Hs.222015 | ESTs Moderately similar to cytokine receptor- | |
| | | | | | | | | like factor 2 cytokine receptor CRL2 precusor | |
| | | | | | | | | [Homo sapiens] | |
| 485 | 523 | AI823792 | lung | primary | 7 | p13 | Hs.301005 | histone H2A.F/Z variant | |
| 486 | 533 | AK025276 | lung | primary | 7 | p13 | Hs.306791 | Homo sapiens cDNA: FLJ21623 fis clone | |
| | | | | | | | | COL07915 | |
| 487 | 534 | AL137266 | lung | primary | 7 | p13 | Hs.332520 | Homo sapiens mRNA cDNA DKFZp434A1014 | |
| | | | | | | | | (from clone DKFZp434A1014) partial cds | |
| 488 | 542 | BC004903 | lung | primary | 7 | p13 | Hs.9960 | hypothetical protein MGC4607 | |
| 489 | 546 | AF192523 | lung | primary | 7 | p13 | Hs.47701 | NPC1 (Niemann-Pick disease type C1 gene)- | |
| | | | | | | | | like 1 | |
| 490 | 550 | AW194730 | lung | primary | 7 | p13 | Hs.9075 | serine/threonine kinase 17a (apoptosis- | |
| | | | | | | | | inducing) | |
| 491 | 541 | BC000769 | lung | primary | 7 | p14.1 | Hs.59594 | hypothetical protein MGC2821 | |
| 492 | 445 | U97188 | lung | primary | 7 | p15.3 | Hs.79440 | IGF-II mRNA-binding protein 3 | |
| 493 | 465 | AI910524 | lung | primary | 7 | p15.3 | Hs.87385 | hypothetical protein BC012331 | |
| 494 | 471 | AI806483 | lung | primary | 7 | p15.3 | Hs.108931 | membrane protein palmitoylated 6 (MAGUK | |
| | | | | | | | | p55 subfamily member 6) | |
| 495 | 494 | AW402635 | lung | primary | 7 | q22.1 | Hs.375569 | DNA directed RNA polymerase II polypeptide | |
| | | | | | | | | J-related gene | |
| 496 | 475 | AI922792 | lung | primary | 8 | NULL | Hs.239784 | scribble | |
| 497 | 489 | R51273 | lung | primary | 8 | q12.2 | Hs.250502 | carbonic anhydrase VIII | |
| 498 | 500 | BE465243 | lung | primary | 8 | q13.2 | Hs.12664 | ESTs | |
| 499 | 503 | AA132172 | lung | primary | 8 | q13.2 | Hs.19107 | ESTs | |
| 500 | 549 | AA203476 | lung | primary | 8 | q13.2 | Hs.252587 | pituitary tumor-transforming 1 | |
| 501 | 555 | AF232217 | lung | primary | 8 | q13.3 | NULL | unknown | |
| 502 | 556 | AF130055 | lung | primary | 8 | q13.3 | NULL | unknown | |
| 503 | 463 | BF002104 | lung | primary | 8 | q21.11 | Hs.168950 | Homo sapiens mRNA cDNA DKFZp566A1046 | |
| | | | | | | | | (from clone DKFZp566A1046) | |
| 504 | 507 | AI335223 | lung | primary | 8 | q21.11 | Hs.133293 | ESTs | |
| 505 | 512 | AI370381 | lung | primary | 8 | q21.11 | Hs.128841 | ESTs | |
| 506 | 529 | AK024242 | lung | primary | 8 | q21.11 | Hs.296753 | Homo sapiens cDNA FLJ14180 fis clone | |
| | | | | | | | | NT2RP2003799 | |
| 507 | 530 | AI701468 | lung | primary | 8 | q21.11 | Hs.60681 | Homo sapiens cDNA FLJ34367 fis clone | |
| | | | | | | | | FEBRA2016621 | |
| 508 | 480 | BG389015 | lung | primary | 8 | q21.13 | Hs.2384 | tumor protein D52 | |
| 509 | 499 | AA479492 | lung | primary | 8 | q21.13 | Hs.184387 | ESTs | |
| 510 | 488 | U07969 | lung | primary | 8 | q22.1 | Hs.89436 | cadherin 17 LI cadherin (liver-intestine) | |
| 511 | 491 | AF091433 | lung | primary | 8 | q22.1 | Hs.30464 | cyclin E2 | |
| 512 | 490 | AA584310 | lung | primary | 8 | q22.3 | Hs.283713 | collagen triple helix repeat containing 1 | |
| 513 | 505 | AA904882 | lung | primary | 8 | q22.3 | Hs.130107 | ESTs | |
| 514 | 543 | AA451665 | lung | primary | 8 | q24.13 | Hs.222088 | hypothetical protein MGC5254 | |
| 515 | 493 | W03103 | lung | primary | 8 | q24.22 | Hs.10669 | development and differentiation enhancing | |
| | | | | | | | | factor 1 | |
| 516 | 518 | BF055351 | lung | primary | 8 | q24.22 | Hs.20247 | ESTs Weakly similar to hypothetical protein | |
| | | | | | | | | FLJ20489 [Homo sapiens] [H. sapiens] | |
| 517 | 544 | BF941325 | lung | primary | 8 | q24.22 | Hs.15611 | KIAA1485 protein | |
| 518 | 535 | AW137073 | lung | primary | 8 | q24.23 | Hs.176669 | Homo sapiens mRNA cDNA DKFZp451M139 | |
| | | | | | | | | (from clone DKFZp451M139) | |
| 519 | 537 | AA447947 | lung | primary | 12 | p11.22 | Hs.227591 | hypothetical protein FLJ11088 | |
| 520 | 456 | R91766 | lung | primary | 12 | p11.23 | Hs.173074 | DKFZP564O1863 protein | |
| 521 | 466 | AF274950 | lung | primary | 12 | p11.23 | Hs.22595 | hypothetical protein FLJ10637 | |
| 522 | 470 | AI334297 | lung | primary | 12 | p11.23 | Hs.51743 | KIAA1340 protein | |
| 523 | 476 | AW779556 | lung | primary | 12 | p11.23 | Hs.184523 | serine/threonine kinase 38 like | |
| 524 | 478 | AI688580 | lung | primary | 12 | p11.23 | Hs.286145 | SRB7 suppressor of RNA polymerase B | |
| | | | | | | | | homolog (yeast) | |
| 525 | 483 | AF161472 | lung | primary | 12 | p11.23 | NULL | unknown | |
| 526 | 504 | BF724206 | lung | primary | 12 | p11.23 | Hs.221024 | ESTs | |
| 527 | 525 | AL118653 | lung | primary | 12 | p11.23 | Hs.284270 | Homo sapiens cDNA FLJ11335 fis clone | |
| | | | | | | | | PLACE1010630 | |
| 528 | 531 | AI652982 | lung | primary | 12 | p11.23 | Hs.111583 | Homo sapiens cDNA FLJ34764 fis clone | |
| | | | | | | | | NT2NE2002311 | |
| 529 | 553 | AA127950 | lung | primary | 12 | p11.23 | Hs.222024 | transcription factor BMAL2 | |
| 530 | 449 | AI652662 | lung | primary | 12 | p12.1 | Hs.317432 | branched chain aminotransferase 1 cytosolic | |
| 531 | 460 | AU154905 | lung | primary | 12 | p12.1 | Hs.296734 | Homo sapiens cDNA FLJ13318 fis clone | |
| | | | | | | | | OVARC1001600 | |
| 532 | 461 | AK025615 | lung | primary | 12 | p12.1 | Hs.7567 | Homo sapiens cDNA: FLJ21962 fis clone | |
| | | | | | | | | HEP05564 | |
| 533 | 462 | AA829940 | lung | primary | 12 | p12.1 | Hs.301210 | Homo sapiens mRNA cDNA DKFZp564F2072 | |
| | | | | | | | | (from clone DKFZp564F2072) | |
| 534 | 468 | BE326710 | lung | primary | 12 | p12.1 | Hs.170994 | hypothetical protein MGC10946 | |
| 535 | 484 | AA015609 | lung | primary | 12 | p12.1 | Hs.351221 | v-Ki-ras2 Kirsten rat sarcoma 2 viral oncogene | |
| | | | | | | | | homolog | |
| 536 | 501 | W70242 | lung | primary | 12 | p12.1 | Hs.58086 | ESTs | |
| 537 | 516 | AI242023 | lung | primary | 12 | p12.1 | Hs.137003 | ESTs | |
| 538 | 520 | AI003792 | lung | primary | 12 | p12.1 | Hs.120439 | ethanolamine kinase | |
| 539 | 554 | AA669106 | lung | primary | 12 | p12.2 | Hs.108106 | ubiquitin-like containing PHD and RING finger | |
| | | | | | | | | domains 1 | |
| 540 | 459 | BC003602 | lung | primary | 12 | p12.3 | Hs.36727 | H2A histone family member J | |
| 541 | 487 | AI392836 | lung | primary | 12 | p13.31 | Hs.12045 | C2f protein | |
| 542 | 492 | AI983033 | lung | primary | 12 | p13.31 | Hs.380623 | DEAD/H (Asp-Glu-Ala-Asp/His) box | |
| | | | | | | | | polypeptide 11 (CHL1-like helicase homolog S. cerevisiae) | |
| 543 | 521 | U74612 | lung | primary | 12 | p13.33 | Hs.239 | forkhead box M1 | |
| 544 | 455 | AF213033 | lung | primary | 14 | q22.1 | Hs.84113 | cyclin-dependent kinase inhibitor 3 (CDK2- | |
| | | | | | | | | associated dual specificity phosphatase) | |
| 545 | 447 | AF167438 | lung | primary | 14 | q23.2 | Hs.179817 | androgen-regulated short-chain | |
| | | | | | | | | dehydrogenase/reductase 1 | |
| 546 | 513 | AI146765 | lung | primary | 18 | p11.31 | Hs.373550 | ESTs | |
| 547 | 532 | AW003207 | lung | primary | 18 | p11.31 | Hs.48659 | Homo sapiens cDNA FLJ36057 fis clone | |
| | | | | | | | | TESTI2018475 highly similar to LAMININ | |
| | | | | | | | | ALPHA-1 CHAIN PRECURSOR | |
| 548 | 444 | AF017790 | lung | primary | 18 | p11.32 | Hs.58169 | highly expressed in cancer rich in leucine | |
| | | | | | | | | heptad repeats | |
| 549 | 450 | AB023169 | lung | primary | 20 | p12.2 | Hs.7935 | BTB (POZ) domain containing 3 | |
| 550 | 457 | AI732446 | lung | primary | 20 | p12.2 | Hs.70903 | ESTs | |
| 551 | 479 | D21267 | lung | primary | 20 | p12.2 | Hs.84389 | synaptosomal-associated protein 25 kDa | |
| 552 | 452 | Y00064 | lung | primary | 20 | p12.3 | Hs.2281 | chromogranin B (secretogranin 1) | |
| 553 | 453 | AI096882 | lung | primary | 20 | p13 | Hs.135056 | chromosome 20 open reading frame 139 | |
| 554 | 454 | AI949781 | lung | primary | 20 | p13 | Hs.26802 | chromosome 20 open reading frame 97 | |
| 555 | 477 | AI924533 | lung | primary | 20 | p13 | Hs.105607 | solute carrier family 4 sodium bicarbonate | |
| | | | | | | | | transporter-like member 11 | |
| 556 | 552 | U85658 | lung | primary | 20 | q13.31 | Hs.61796 | transcription factor AP-2 gamma (activating | |
| | | | | | | | | enhancer binding protein 2 gamma) | |
| 557 | 654 | AW151887 | lung | metastatic | 1 | p22.3 | Hs.169939 | heparan sulfate 2-O-sulfotransferase 1 | |
| 558 | 651 | BE645144 | lung | metastatic | 1 | p31.1 | Hs.374411 | ESTs Moderately similar to hypothetical | |
| | | | | | | | | protein FLJ20378 [Homo sapiens] [H. sapiens] | |
| 559 | 619 | AI810054 | lung | metastatic | 1 | p31.2 | Hs.133260 | hypothetical protein FLJ20354 | |
| 560 | 629 | N32508 | lung | metastatic | 1 | p31.2 | Hs.8107 | G-protein gamma-12 subunit | |
| 561 | 636 | BC002488 | lung | metastatic | 1 | p31.2 | Hs.165998 | PAI-1 mRNA-binding protein | |
| 562 | 628 | AA618420 | lung | metastatic | 1 | p32.1 | Hs.299254 | Homo sapiens cDNA: FLJ23597 fis clone | |
| | | | | | | | | LNG15281 | |
| 563 | 627 | AW140098 | lung | metastatic | 1 | p32.3 | Hs.25821 | Fas (TNFRSF6) associated factor 1 | |
| 564 | 648 | AW409848 | lung | metastatic | 1 | p32.3 | Hs.13036 | DKFZP727A071 protein | |
| 565 | 637 | AF151063 | lung | metastatic | 1 | p34.1 | NULL | unknown | |
| 566 | 600 | AA926959 | lung | metastatic | 1 | q21.3 | Hs.77550 | p53-regulated DDA3 | |
| 567 | 576 | AI766666 | lung | metastatic | 1 | q22 | Hs.374850 | apolipoprotein A-I binding protein | |
| 568 | 588 | AI690773 | lung | metastatic | 1 | q22 | Hs.133294 | ESTs | |
| 569 | 601 | AI739071 | lung | metastatic | 1 | q22 | Hs.158515 | hypothetical protein MGC13038 | |
| 570 | 561 | AF326731 | lung | metastatic | 1 | q23.3 | Hs.234545 | cell division cycle associated 1 | |
| 571 | 562 | D78335 | lung | metastatic | 1 | q23.3 | Hs.75939 | uridine monophosphate kinase | |
| 572 | 558 | AA182412 | lung | metastatic | 1 | q25.3 | Hs.32058 | chromosome 1 open reading frame 19 | |
| 573 | 599 | AA725362 | lung | metastatic | 2 | p11.1 | NULL | unknown | |
| 574 | 592 | AI990317 | lung | metastatic | 2 | p13.1 | Hs.154672 | methylene tetrahydrofolate dehydrogenase | |
| | | | | | | | | (NAD+ dependent) methenyltetrahydrofolate | |
| | | | | | | | | cyclohydrolase | |
| 575 | 603 | AI191897 | lung | metastatic | 2 | p16.2 | Hs.105223 | Homo sapiens Similar to RIKEN cDNA | |
| | | | | | | | | 2510006C20 gene clone MGC: 24001 | |
| | | | | | | | | IMAGE: 4050858 mRNA complete cds | |
| 576 | 583 | BC001886 | lung | metastatic | 2 | p25.1 | Hs.75319 | ribonucleotide reductase M2 polypeptide | |
| 577 | 605 | H24953 | lung | metastatic | 2 | q13 | NULL | unknown | |
| 578 | 575 | AA749314 | lung | metastatic | 2 | q31.1 | Hs.333893 | cell division cycle associated 7 | |
| 579 | 579 | AA868748 | lung | metastatic | 5 | p15.1 | Hs.125249 | ESTs | |
| 580 | 589 | AI439141 | lung | metastatic | 6 | p23 | Hs.261023 | hypothetical protein FLJ20958 | |
| 581 | 606 | AU156822 | lung | metastatic | 7 | p11.2 | Hs.287577 | Homo sapiens cDNA FLJ13503 fis clone | |
| | | | | | | | | PLACE1004838 | |
| 582 | 607 | U48722 | lung | metastatic | 7 | p11.2 | NULL | unknown | |
| 583 | 609 | AA768884 | lung | metastatic | 7 | p11.2 | Hs.140489 | Homo sapiens cDNA FLJ25559 fis clone | |
| | | | | | | | | JTH02834 | |
| 584 | 610 | AK000106 | lung | metastatic | 7 | p11.2 | Hs.272227 | Homo sapiens cDNA FLJ20099 fis clone | |
| | | | | | | | | COL04544 | |
| 585 | 613 | AU147861 | lung | metastatic | 7 | p11.2 | Hs.188082 | Homo sapiens cDNA FLJ12308 fis clone | |
| | | | | | | | | MAMMA1001931 | |
| 586 | 616 | BE737030 | lung | metastatic | 7 | p11.2 | Hs.82916 | chaperonin containing TCP1 subunit 6A (zeta | |
| | | | | | | | | 1) | |
| 587 | 622 | AW157070 | lung | metastatic | 7 | p11.2 | Hs.77432 | epidermal growth factor receptor | |
| | | | | | | | | (erythroblastic leukemia viral (v-erb-b) | |
| | | | | | | | | oncogene homolog avian) | |
| 588 | 639 | BE878463 | lung | metastatic | 7 | p11.2 | Hs.279898 | Homo sapiens cDNA: FLJ23165 fis clone | |
| | | | | | | | | LNG09846 | |
| 589 | 557 | AK023208 | lung | metastatic | 7 | p14.2 | Hs.62180 | anillin actin binding protein (scraps homolog | |
| | | | | | | | | Drosophila) | |
| 590 | 560 | U97188 | lung | metastatic | 7 | p15.3 | Hs.79440 | IGF-II mRNA-binding protein 3 | |
| 591 | 577 | AI910524 | lung | metastatic | 7 | p15.3 | Hs.87385 | hypothetical protein BC012331 | |
| 592 | 590 | AI806483 | lung | metastatic | 7 | p15.3 | Hs.108931 | membrane protein palmitoylated 6 (MAGUK | |
| | | | | | | | | p55 subfamily member 6) | |
| 593 | 617 | AL136770 | lung | metastatic | 7 | q21.13 | Hs.258576 | claudin 12 | |
| 594 | 635 | BF680588 | lung | metastatic | 7 | q21.13 | Hs.118258 | Homo sapiens cDNA: FLJ23160 fis clone | |
| | | | | | | | | LNG09682 | |
| 595 | 618 | U19348 | lung | metastatic | 7 | q31.2 | NULL | unknown | |
| 596 | 638 | BG170541 | lung | metastatic | 7 | q31.2 | Hs.285754 | met proto-oncogene (hepatocyte growth factor | |
| | | | | | | | | receptor) | |
| 597 | 641 | AI632244 | lung | metastatic | 7 | q32.1 | Hs.233694 | putative methyltransferase | |
| 598 | 653 | AI964022 | lung | metastatic | 7 | q33 | Hs.107394 | secretory protein SEC8 | |
| 599 | 593 | AI922792 | lung | metastatic | 8 | NULL | Hs.239784 | scribble | |
| 600 | 644 | AA723810 | lung | metastatic | 8 | NULL | Hs.69517 | cDNA for differentially expressed CO16 gene | |
| 601 | 621 | BF059124 | lung | metastatic | 8 | q12.3 | Hs.29419 | ESTs | |
| 602 | 631 | AA543030 | lung | metastatic | 8 | q12.3 | Hs.152409 | ESTs | |
| 603 | 632 | AF289489 | lung | metastatic | 8 | q12.3 | Hs.283664 | aspartate beta-hydroxylase | |
| 604 | 646 | AW663544 | lung | metastatic | 8 | q13.1 | Hs.85524 | ring finger protein 29 | |
| 605 | 581 | BF002104 | lung | metastatic | 8 | q21.11 | Hs.168950 | Homo sapiens mRNA cDNA DKFZp566A1046 | |
| | | | | | | | | (from clone DKFZp566A1046) | |
| 606 | 612 | AI916600 | lung | metastatic | 8 | q21.11 | Hs.121194 | Homo sapiens cDNA: FLJ21569 fis clone | |
| | | | | | | | | COL06508 | |
| 607 | 623 | AI625741 | lung | metastatic | 8 | q21.11 | Hs.21275 | hypothetical protein FLJ11011 | |
| 608 | 630 | AW150720 | lung | metastatic | 8 | q21.11 | Hs.356086 | ESTs Weakly similar to retinal short-chain | |
| | | | | | | | | dehydrogenase/reductase retSDR2 [Homo | |
| | | | | | | | | sapiens] [H. sapiens] | |
| 609 | 645 | N89607 | lung | metastatic | 8 | q21.11 | Hs.184693 | transcription elongation factor B (SIII) | |
| | | | | | | | | polypeptide 1 (15 kDa elongin C) | |
| 610 | 650 | W46994 | lung | metastatic | 8 | q21.11 | Hs.96870 | staufen RNA binding protein homolog 2 | |
| | | | | | | | | (Drosophila) | |
| 611 | 563 | BG389015 | lung | metastatic | 8 | q21.13 | Hs.2384 | tumor protein D52 | |
| 612 | 633 | AK000049 | lung | metastatic | 8 | q21.13 | Hs.183861 | hypothetical protein MGC22825 | |
| 613 | 634 | AK024296 | lung | metastatic | 8 | q21.13 | Hs.237146 | zinc finger protein RINZF | |
| 614 | 656 | AL039862 | lung | metastatic | 8 | q24.21 | Hs.49136 | Homo sapiens cDNA FLJ23705 fis clone | |
| | | | | | | | | HEP11066 | |
| 615 | 611 | M26095 | lung | metastatic | 11 | p15.2 | Hs.37058 | calcitonin/calcitonin-related polypeptide alpha | |
| 616 | 565 | AF256215 | lung | metastatic | 12 | p11.23 | Hs.222024 | transcription factor BMAL2 | |
| 617 | 566 | AI569851 | lung | metastatic | 12 | p11.23 | Hs.22595 | hypothetical protein FLJ10637 | |
| 618 | 573 | AF161472 | lung | metastatic | 12 | p11.23 | NULL | unknown | |
| 619 | 574 | U46837 | lung | metastatic | 12 | p11.23 | Hs.286145 | SRB7 suppressor of RNA polymerase B | |
| | | | | | | | | homolog (yeast) | |
| 620 | 580 | R91766 | lung | metastatic | 12 | p11.23 | Hs.173074 | DKFZP564O1863 protein | |
| 621 | 584 | AI334297 | lung | metastatic | 12 | p11.23 | Hs.51743 | KIAA1340 protein | |
| 622 | 585 | AW779556 | lung | metastatic | 12 | p11.23 | Hs.184523 | serine/threonine kinase 38 like | |
| 623 | 586 | BF540749 | lung | metastatic | 12 | p11.23 | Hs.111583 | Homo sapiens cDNA FLJ34764 fis clone | |
| | | | | | | | | NT2NE2002311 | |
| 624 | 587 | BC005176 | lung | metastatic | 12 | p11.23 | Hs.10071 | seven transmembrane protein TM7SF3 | |
| 625 | 564 | AI652662 | lung | metastatic | 12 | p12.1 | Hs.317432 | branched chain aminotransferase 1 cytosolic | |
| 626 | 568 | AK025615 | lung | metastatic | 12 | p12.1 | Hs.7567 | Homo sapiens cDNA: FLJ21962 fis clone | |
| | | | | | | | | HEP05564 | |
| 627 | 570 | BE326710 | lung | metastatic | 12 | p12.1 | Hs.170994 | hypothetical protein MGC10946 | |
| 628 | 595 | AA829940 | lung | metastatic | 12 | p12.1 | Hs.301210 | Homo sapiens mRNA cDNA DKFZp564F2072 | |
| | | | | | | | | (from clone DKFZp564F2072) | |
| 629 | 597 | AA015609 | lung | metastatic | 12 | p12.1 | Hs.351221 | v-Ki-ras2 Kirsten rat sarcoma 2 viral oncogene | |
| | | | | | | | | homolog | |
| 630 | 604 | AU154905 | lung | metastatic | 12 | p12.1 | Hs.296734 | Homo sapiens cDNA FLJ13318 fis clone | |
| | | | | | | | | OVARC1001600 | |
| 631 | 571 | AK025578 | lung | metastatic | 12 | p12.2 | Hs.108106 | ubiquitin-like containing PHD and RING finger | |
| | | | | | | | | domains 1 | |
| 632 | 578 | BC003602 | lung | metastatic | 12 | p12.3 | Hs.36727 | H2A histone family member J | |
| 633 | 643 | AI743489 | lung | metastatic | 12 | p13.1 | Hs.322679 | Homo sapiens cDNA FLJ36082 fis clone | |
| | | | | | | | | TESTI2019998 | |
| 634 | 642 | AA102574 | lung | metastatic | 14 | q12 | Hs.8858 | bromodomain adjacent to zinc finger domain | |
| | | | | | | | | 1A | |
| 635 | 620 | AI953589 | lung | metastatic | 14 | q13.1 | Hs.146134 | ESTs | |
| 636 | 608 | AW268365 | lung | metastatic | 14 | q21.3 | Hs.25740 | ERO1-like (S. cerevisiae) | |
| 637 | 626 | BC006117 | lung | metastatic | 14 | q21.3 | Hs.222021 | hypothetical protein FLJ12618 | |
| 638 | 655 | AJ292969 | lung | metastatic | 14 | q21.3 | Hs.288906 | WW45 protein | |
| 639 | 567 | AF213033 | lung | metastatic | 14 | q22.1 | Hs.84113 | cyclin-dependent kinase inhibitor 3 (CDK2- | |
| | | | | | | | | associated dual specificity phosphatase) | |
| 640 | 652 | BC005359 | lung | metastatic | 14 | q22.1 | Hs.151413 | glia maturation factor beta | |
| 641 | 614 | AI985034 | lung | metastatic | 14 | q23.1 | Hs.2704 | glutathione peroxidase 2 (gastrointestinal) | |
| 642 | 624 | AI554514 | lung | metastatic | 14 | q23.1 | Hs.97849 | ESTs | |
| 643 | 569 | AF167438 | lung | metastatic | 14 | q23.2 | Hs.179817 | androgen-regulated short-chain | |
| | | | | | | | | dehydrogenase/reductase 1 | |
| 644 | 625 | AI654093 | lung | metastatic | 14 | q23.2 | Hs.43397 | Homo sapiens cDNA FLJ37574 fis clone | |
| | | | | | | | | BRCOC2003100 | |
| 645 | 647 | BE465894 | lung | metastatic | 14 | q24.2 | Hs.98365 | hypothetical protein FLJ39091 | |
| 646 | 615 | AI969102 | lung | metastatic | 14 | q32.11 | Hs.172216 | chromogranin A (parathyroid secretory protein | |
| | | | | | | | | 1) | |
| 647 | 640 | AI656232 | lung | metastatic | 14 | q32.11 | Hs.90034 | hypothetical protein FLJ21916 | |
| 648 | 649 | AI670847 | lung | metastatic | 14 | q32.12 | Hs.374662 | Homo sapiens cDNA FLJ40513 fis clone | |
| | | | | | | | | TESTI2046456 | |
| 649 | 559 | AF017790 | lung | metastatic | 18 | p11.32 | Hs.58169 | highly expressed in cancer rich in leucine | |
| | | | | | | | | heptad repeats | |
| 650 | 591 | D21267 | lung | metastatic | 20 | p12.2 | Hs.84389 | synaptosomal-associated protein 25 kDa | |
| 651 | 598 | AI732446 | lung | metastatic | 20 | p12.2 | Hs.70903 | ESTs | |
| 652 | 602 | AB023169 | lung | metastatic | 20 | p12.2 | Hs.7935 | BTB (POZ) domain containing 3 | |
| 653 | 572 | Y00064 | lung | metastatic | 20 | p12.3 | Hs.2281 | chromogranin B (secretogranin 1) | |
| 654 | 582 | AI949781 | lung | metastatic | 20 | p13 | Hs.26802 | chromosome 20 open reading frame 97 | |
| 655 | 594 | AF336127 | lung | metastatic | 20 | p13 | Hs.105607 | solute carrier family 4 sodium bicarbonate | |
| | | | | | | | | transporter-like member 11 | |
| 656 | 596 | AI096882 | lung | metastatic | 20 | p13 | Hs.135056 | chromosome 20 open reading frame 139 | |
| 657 | 662 | AK000490 | prostate | primary | 1 | p31.2 | Hs.133260 | hypothetical protein FLJ20354 | |
| 658 | 659 | AW271106 | prostate | primary | 1 | q22 | Hs.133294 | ESTs | |
| 659 | 660 | AI053741 | prostate | primary | 1 | q22 | Hs.133294 | ESTs | |
| 660 | 663 | AA830844 | prostate | primary | 1 | q23.2 | Hs.127310 | kinase interacting with leukemia-associated | |
| | | | | | | | | gene (stathmin) | |
| 661 | 657 | AF326731 | prostate | primary | 1 | q23.3 | Hs.234545 | cell division cycle associated 1 | |
| 662 | 661 | AB032931 | prostate | primary | 1 | q32.1 | Hs.5199 | HSPC150 protein similar to ubiquitin- | |
| | | | | | | | | conjugating enzyme | |
| 663 | 658 | U30872 | prostate | primary | 1 | q32.3 | Hs.77204 | centromere protein F 350/400 ka (mitosin) | |
| 664 | 684 | AI492879 | prostate | primary | 2 | p25.1 | Hs.75319 | ribonucleotide reductase M2 polypeptide | |
| 665 | 683 | N21131 | prostate | primary | 2 | q37.3 | Hs.42949 | hairy and enhancer of split 6 (Drosophila) | |
| 666 | 690 | BE407516 | prostate | primary | 5 | q13.2 | Hs.23960 | cyclin B1 | |
| 667 | 691 | U70370 | prostate | primary | 5 | q31.1 | Hs.84136 | paired-like homeodomain transcription factor 1 | |
| 668 | 692 | BE794699 | prostate | primary | 6 | p21.2 | Hs.284207 | hypothetical protein BC003515 | |
| 669 | 694 | AI343467 | prostate | primary | 7 | p14.1 | Hs.28792 | Homo sapiens cDNA FLJ11041 fis clone | |
| | | | | | | | | PLACE1004405 | |
| 670 | 695 | M13436 | prostate | primary | 7 | p14.1 | Hs.727 | inhibin beta A (activin A activin AB alpha | |
| | | | | | | | | polypeptide) | |
| 671 | 693 | AK023208 | prostate | primary | 7 | p14.2 | Hs.62180 | anillin actin binding protein (scraps homolog | |
| | | | | | | | | Drosophila) | |
| 672 | 698 | AI932328 | prostate | primary | 8 | p21.1 | Hs.104741 | T-LAK cell-originated protein kinase | |
| 673 | 697 | AA203476 | prostate | primary | 8 | q13.2 | Hs.252587 | pituitary tumor-transforming 1 | |
| 674 | 696 | AI925583 | prostate | primary | 8 | q24.13 | Hs.222088 | hypothetical protein MGC5254 | |
| 675 | 700 | BE544837 | prostate | primary | 9 | q33.2 | Hs.352417 | Homo sapiens Similar to RIKEN cDNA | |
| | | | | | | | | 3321402G02 gene clone MGC: 23929 | |
| | | | | | | | | IMAGE: 4807540 mRNA complete cds | |
| 676 | 699 | AI983261 | prostate | primary | 9 | q34.3 | Hs.323445 | ESTs Weakly similar to T2D3_HUMAN | |
| | | | | | | | | Transcription initiation factor TFIID 135 kDa | |
| | | | | | | | | subunit (TAFII-135) (TAFII135) | |
| 677 | 664 | X05360 | prostate | primary | 10 | q21.2 | Hs.334562 | cell division cycle 2 G1 to S and G2 to M | |
| 678 | 665 | AI674163 | prostate | primary | 10 | q23.33 | Hs.14559 | hypothetical protein FLJ10540 | |
| 679 | 666 | BE614410 | prostate | primary | 11 | q13.1 | Hs.23044 | similar to RIKEN cDNA 2610036L13 | |
| 680 | 667 | U74612 | prostate | primary | 12 | p13.33 | Hs.239 | forkhead box M1 | |
| 681 | 668 | R61322 | prostate | primary | 12 | q24.31 | Hs.204166 | Human clone 295 5cM region surrounding | |
| | | | | | | | | hepatocyte nuclear factor-1a/MODY3 mRNA | |
| 682 | 669 | L25876 | prostate | primary | 14 | q22.1 | Hs.84113 | cyclin-dependent kinase inhibitor 3 (CDK2- | |
| | | | | | | | | associated dual specificity phosphatase) | |
| 683 | 670 | U65410 | prostate | primary | 14 | q23.1 | Hs.79078 | MAD2 mitotic arrest deficient-like 1 (yeast) | |
| 684 | 671 | AL080146 | prostate | primary | 15 | q21.3 | Hs.194698 | cyclin B2 | |
| 685 | 672 | D14657 | prostate | primary | 15 | q22.2 | Hs.81892 | KIAA0101 gene product | |
| 686 | 674 | AB018009 | prostate | primary | 16 | NULL | Hs.184601 | solute carrier family 7 (cationic amino acid | |
| | | | | | | | | transporter y+ system) member 5 | |
| 687 | 673 | AI819340 | prostate | primary | 16 | p13.3 | Hs.13561 | hypothetical protein MGC4692 | |
| 688 | 678 | BE328850 | prostate | primary | 17 | q11.2 | Hs.348504 | hypothetical protein BC014072 | |
| 689 | 679 | AF063308 | prostate | primary | 17 | q11.2 | Hs.16244 | mitotic spindle coiled-coil related protein | |
| 690 | 676 | AW003286 | prostate | primary | 17 | q21.31 | Hs.370428 | ESTs Moderately similar to TP2A_HUMAN | |
| | | | | | | | | DNA topoisomerase II alpha isozyme | |
| | | | | | | | | [H. sapiens] | |
| 691 | 680 | AI375913 | prostate | primary | 17 | q21.31 | Hs.156346 | topoisomerase (DNA) II alpha 170 kDa | |
| 692 | 681 | L47276 | prostate | primary | 17 | q21.31 | NULL | unknown | |
| 693 | 677 | BG165011 | prostate | primary | 17 | q23.2 | Hs.165909 | ESTs Weakly similar to hypothetical protein | |
| | | | | | | | | FLJ20489 [Homo sapiens] [H. sapiens] | |
| 694 | 675 | BF056791 | prostate | primary | 17 | q23.3 | Hs.87507 | ESTs | |
| 695 | 682 | AA719022 | prostate | primary | 19 | q13.43 | Hs.288549 | ubiquitin UBF-fl | |
| 696 | 686 | D80008 | prostate | primary | 20 | p11.21 | Hs.36232 | KIAA0186 gene product | |
| 697 | 685 | AF098158 | prostate | primary | 20 | q11.1 | Hs.9329 | chromosome 20 open reading frame 1 | |
| 698 | 688 | U73379 | prostate | primary | 20 | q13.12 | Hs.93002 | ubiquitin-conjugating enzyme E2C | |
| 699 | 687 | AF011468 | prostate | primary | 20 | q13.31 | Hs.250822 | serine/threonine kinase 6 | |
| 700 | 701 | T77624 | prostate | primary | 21 | q22.13 | Hs.79375 | holocarboxylase synthetase (biotin-[proprionyl- | |
| | | | | | | | | Coenzyme A-carboxylase (ATP-hydrolysing)] | |
| | | | | | | | | ligase) | |
| 701 | 689 | AI381686 | prostate | primary | 22 | q13.2 | Hs.208912 | hypothetical protein MGC861 | |
| 702 | 723 | AA630330 | prostate | metastatic | 1 | q21.2 | Hs.89545 | proteasome (prosome macropain) subunit beta | |
| | | | | | | | | type 4 | |
| 703 | 771 | AW271106 | prostate | metastatic | 1 | q22 | Hs.133294 | ESTs | |
| 704 | 773 | AI690773 | prostate | metastatic | 1 | q22 | Hs.133294 | ESTs | |
| 705 | 803 | AI766666 | prostate | metastatic | 1 | q22 | Hs.374850 | apolipoprotein A-I binding protein | |
| 706 | 739 | AI249980 | prostate | metastatic | 1 | q23.2 | Hs.127310 | kinase interacting with leukemia-associated | |
| | | | | | | | | gene (stathmin) | |
| 707 | 798 | AI015982 | prostate | metastatic | 1 | q23.3 | Hs.234545 | cell division cycle associated 1 | |
| 708 | 745 | H62656 | prostate | metastatic | 1 | q24.3 | Hs.300893 | hypothetical protein MGC17528 | |
| 709 | 753 | N29457 | prostate | metastatic | 1 | q31.1 | Hs.117305 | hypothetical gene supported by BC007071 | |
| 710 | 796 | U30872 | prostate | metastatic | 1 | q32.3 | Hs.77204 | centromere protein F 350/400 ka (mitosin) | |
| 711 | 794 | AA151971 | prostate | metastatic | 1 | q42.2 | Hs.334372 | chorionic somatomammotropin hormone 2 | |
| 712 | 748 | AI971357 | prostate | metastatic | 3 | p21.32 | Hs.146170 | hypothetical protein FLJ22969 | |
| 713 | 778 | W24316 | prostate | metastatic | 3 | q12.3 | Hs.173374 | endothelial and smooth muscle cell-derived | |
| | | | | | | | | neuropilin-like protein | |
| 714 | 716 | AI338462 | prostate | metastatic | 3 | q26.1 | Hs.50758 | SMC4 structural maintenance of | |
| | | | | | | | | chromosomes 4-like 1 (yeast) | |
| 715 | 717 | AB019987 | prostate | metastatic | 3 | q26.1 | Hs.50758 | SMC4 structural maintenance of | |
| | | | | | | | | chromosomes 4-like 1 (yeast) | |
| 716 | 740 | AL119157 | prostate | metastatic | 3 | q26.32 | Hs.22941 | KIAA1363 protein | |
| 717 | 777 | BG170335 | prostate | metastatic | 3 | q26.32 | Hs.122579 | epithelial cell transforming sequence 2 | |
| | | | | | | | | oncogene | |
| 718 | 704 | AI968388 | prostate | metastatic | 3 | q26.33 | NULL | unknown | |
| 719 | 724 | AA194529 | prostate | metastatic | 3 | q28 | Hs.74619 | proteasome (prosome macropain) 26S subunit | |
| | | | | | | | | non-ATPase 2 | |
| 720 | 760 | BE256479 | prostate | metastatic | 5 | p14.3 | Hs.79037 | heat shock 60 kDa protein 1 (chaperonin) | |
| 721 | 705 | AI750154 | prostate | metastatic | 5 | p15.1 | NULL | unknown | |
| 722 | 711 | U96131 | prostate | metastatic | 5 | p15.33 | Hs.6566 | thyroid hormone receptor interactor 13 | |
| 723 | 787 | M25753 | prostate | metastatic | 5 | q13.2 | Hs.23960 | cyclin B1 | |
| 724 | 758 | AI369840 | prostate | metastatic | 6 | p21.1 | Hs.374582 | Homo sapiens cDNA FLJ11842 fis clone | |
| | | | | | | | | HEMBA1006652 weakly similar to 60S | |
| | | | | | | | | RIBOSOMAL PROTEIN L7 | |
| 725 | 804 | AK023208 | prostate | metastatic | 7 | p14.2 | Hs.62180 | anillin actin binding protein (scraps homolog | |
| | | | | | | | | Drosophila) | |
| 726 | 752 | AI910524 | prostate | metastatic | 7 | p15.3 | Hs.87385 | hypothetical protein BC012331 | |
| 727 | 721 | L07493 | prostate | metastatic | 7 | p21.3 | Hs.1608 | replication protein A3 14 kDa | |
| 728 | 730 | AL582836 | prostate | metastatic | 7 | q21.3 | Hs.137476 | paternally expressed 10 | |
| 729 | 770 | AI922470 | prostate | metastatic | 7 | q21.3 | Hs.370106 | ESTs Highly similar to asparagine synthetase | |
| | | | | | | | | [Homo sapiens] [H. sapiens] | |
| 730 | 726 | L37127 | prostate | metastatic | 7 | q22.1 | Hs.80475 | polymerase (RNA) II (DNA directed) | |
| | | | | | | | | polypeptide J 13.3 kDa | |
| 731 | 736 | AA193396 | prostate | metastatic | 7 | q31.2 | Hs.285754 | met proto-oncogene (hepatocyte growth factor | |
| | | | | | | | | receptor) | |
| 732 | 768 | AI679933 | prostate | metastatic | 7 | q33 | Hs.369347 | ESTs Weakly similar to hypothetical protein | |
| | | | | | | | | FLJ20378 [Homo sapiens] [H. sapiens] | |
| 733 | 763 | AI571298 | prostate | metastatic | 8 | NULL | Hs.343589 | exosome component Rrp41 | |
| 734 | 732 | AA191576 | prostate | metastatic | 8 | q12.1 | Hs.9614 | nucleophosmin (nucleolar phosphoprotein B23 | |
| | | | | | | | | numatrin) | |
| 735 | 801 | AW001796 | prostate | metastatic | 8 | q12.3 | Hs.283664 | aspartate beta-hydroxylase | |
| 736 | 729 | AA203476 | prostate | metastatic | 8 | q13.2 | Hs.252587 | pituitary tumor-transforming 1 | |
| 737 | 795 | AI525903 | prostate | metastatic | 8 | q13.3 | Hs.118554 | CGI-83 protein | |
| 738 | 710 | AA995715 | prostate | metastatic | 8 | q21.11 | Hs.184693 | transcription elongation factor B (SIII) | |
| | | | | | | | | polypeptide 1 (15 kDa elongin C) | |
| 739 | 782 | BE409290 | prostate | metastatic | 8 | q22.3 | Hs.273344 | DKFZP564O0463 protein | |
| 740 | 788 | AA584310 | prostate | metastatic | 8 | q22.3 | Hs.283713 | collagen triple helix repeat containing 1 | |
| 741 | 769 | BF109660 | prostate | metastatic | 8 | q23.1 | Hs.127286 | ESTs Moderately similar to leucine-rich | |
| | | | | | | | | neuronal protein [Homo sapiens] [H. sapiens] | |
| 742 | 789 | AI802955 | prostate | metastatic | 8 | q23.2 | Hs.195870 | chronic myelogenous leukemia tumor antigen | |
| | | | | | | | | 66 | |
| 743 | 737 | AL117612 | prostate | metastatic | 8 | q24.12 | Hs.76550 | mal T-cell differentiation protein 2 | |
| 744 | 756 | AI880004 | prostate | metastatic | 8 | q24.22 | Hs.356036 | Homo sapiens mRNA cDNA DKFZp666E036 | |
| | | | | | | | | (from clone DKFZp666E036) | |
| 745 | 784 | AI023398 | prostate | metastatic | 8 | q24.22 | Hs.10669 | development and differentiation enhancing | |
| | | | | | | | | factor 1 | |
| 746 | 706 | AA527374 | prostate | metastatic | 8 | q24.23 | NULL | unknown | |
| 747 | 702 | AF067656 | prostate | metastatic | 10 | q21.1 | Hs.42650 | ZW10 interactor | |
| 748 | 799 | AF154332 | prostate | metastatic | 10 | q21.2 | Hs.334562 | cell division cycle 2 G1 to S and G2 to M | |
| 749 | 802 | BC006121 | prostate | metastatic | 10 | q22.1 | Hs.117062 | apoptosis-inducing factor (AIF)-homologous | |
| | | | | | | | | mitochondrion-associated inducer of death | |
| 750 | 728 | K03226 | prostate | metastatic | 10 | q22.2 | Hs.77274 | plasminogen activator urokinase | |
| 751 | 805 | U90339 | prostate | metastatic | 10 | q22.2 | Hs.94382 | adenosine kinase | |
| 752 | 725 | AI198535 | prostate | metastatic | 10 | q22.3 | Hs.89463 | potassium large conductance calcium- | |
| | | | | | | | | activated channel subfamily M alpha member 1 | |
| 753 | 734 | N27428 | prostate | metastatic | 10 | q23.31 | Hs.240 | M-phase phosphoprotein 1 | |
| 754 | 751 | AI674163 | prostate | metastatic | 10 | q23.33 | Hs.14559 | hypothetical protein FLJ10540 | |
| 755 | 718 | BE614410 | prostate | metastatic | 11 | q13.1 | Hs.23044 | similar to RIKEN cDNA 2610036L13 | |
| 756 | 761 | BG251266 | prostate | metastatic | 11 | q13.1 | Hs.283565 | FOS-like antigen 1 | |
| 757 | 733 | AA621983 | prostate | metastatic | 11 | q13.3 | Hs.116051 | myeloma overexpressed gene (in a subset of | |
| | | | | | | | | t(11 14) positive multiple myelomas) | |
| 758 | 722 | U82984 | prostate | metastatic | 12 | q13.12 | Hs.23900 | Rac GTPase activating protein 1 | |
| 759 | 747 | AF091087 | prostate | metastatic | 12 | q13.12 | Hs.206501 | hypothetical protein from clone 643 | |
| 760 | 754 | AI936946 | prostate | metastatic | 12 | q13.12 | Hs.121973 | Homo sapiens clone MGC: 20874 | |
| | | | | | | | | IMAGE: 4547239 mRNA complete cds | |
| 761 | 707 | AL118633 | prostate | metastatic | 12 | q13.13 | Hs.151678 | UDP-N-acetyl-alpha-D- | |
| | | | | | | | | galactosamine:polypeptide N- | |
| | | | | | | | | acetylgalactosaminyltransferase 6 (GalNAc- | |
| | | | | | | | | T6) | |
| 762 | 741 | X81420 | prostate | metastatic | 12 | q13.13 | Hs.32952 | keratin hair basic 1 | |
| 763 | 762 | D79987 | prostate | metastatic | 12 | q13.13 | Hs.153479 | extra spindle poles like 1 (S. cerevisiae) | |
| 764 | 727 | AF025840 | prostate | metastatic | 14 | q21.2 | Hs.99185 | polymerase (DNA directed) epsilon 2 (p59 | |
| | | | | | | | | subunit) | |
| 765 | 744 | AA648933 | prostate | metastatic | 14 | q21.2 | Hs.374811 | hypothetical protein MGC20689 | |
| 766 | 749 | BC006117 | prostate | metastatic | 14 | q21.3 | Hs.222021 | hypothetical protein FLJ12618 | |
| 767 | 750 | AI924794 | prostate | metastatic | 14 | q21.3 | Hs.27931 | hypothetical protein FLJ10607 similar to | |
| | | | | | | | | glucosamine-phosphate N-acetyltransferase | |
| 768 | 776 | AW268365 | prostate | metastatic | 14 | q21.3 | Hs.25740 | ERO1-like (S. cerevisiae) | |
| 769 | 780 | D13633 | prostate | metastatic | 14 | q22.1 | Hs.77695 | Drosophila discs large-1 tumor supressor-like | |
| 770 | 785 | L25876 | prostate | metastatic | 14 | q22.1 | Hs.84113 | cyclin-dependent kinase inhibitor 3 (CDK2- | |
| | | | | | | | | associated dual specificity phosphatase) | |
| 771 | 766 | AI417084 | prostate | metastatic | 14 | q22.2 | Hs.301231 | ESTs Weakly similar to PSA3_HUMAN | |
| | | | | | | | | Proteasome subunit alpha type 3 (Proteasome | |
| | | | | | | | | component C8) (Macropain | |
| 772 | 735 | J04031 | prostate | metastatic | 14 | q23.1 | Hs.172665 | methylenetetrahydrofolate dehydrogenase | |
| | | | | | | | | (NADP+ dependent) methenyltetrahydrofolate | |
| | | | | | | | | cyclohydrolase formyltetrahydrofolate | |
| | | | | | | | | synthetase | |
| 773 | 738 | U65410 | prostate | metastatic | 14 | q23.1 | Hs.79078 | MAD2 mitotic arrest deficient-like 1 (yeast) | |
| 774 | 731 | AA926959 | prostate | metastatic | 14 | q32.12 | Hs.77550 | p53-regulated DDA3 | |
| 775 | 764 | AL080102 | prostate | metastatic | 14 | q32.2 | Hs.334810 | eukaryotic translation initiation factor 5 | |
| 776 | 779 | BF000332 | prostate | metastatic | 14 | q32.2 | Hs.7720 | dynein cytoplasmic heavy polypeptide 1 | |
| 777 | 786 | AI525727 | prostate | metastatic | 14 | q32.2 | Hs.38205 | cyclin-dependent kinase 2-interacting protein | |
| 778 | 800 | AI761729 | prostate | metastatic | 14 | q32.2 | Hs.12908 | CDC42 binding protein kinase beta (DMPK- | |
| | | | | | | | | like) | |
| 779 | 746 | T65554 | prostate | metastatic | 14 | q32.31 | Hs.317821 | hypothetical protein MGC13251 | |
| 780 | 755 | H04885 | prostate | metastatic | 14 | q32.31 | Hs.72363 | Homo sapiens clone MGC: 16771 | |
| | | | | | | | | IMAGE: 3907551 mRNA complete cds | |
| 781 | 797 | AI684508 | prostate | metastatic | 14 | q32.31 | Hs.34045 | cell division cycle associated 4 | |
| 782 | 715 | U81800 | prostate | metastatic | 17 | NULL | Hs.85838 | solute carrier family 16 (monocarboxylic acid | |
| | | | | | | | | transporters) member 3 | |
| 783 | 774 | AI292123 | prostate | metastatic | 17 | NULL | Hs.201390 | ESTs | |
| 784 | 743 | AI458014 | prostate | metastatic | 17 | q22 | Hs.283558 | hypothetical protein PRO1855 | |
| 785 | 775 | AA564822 | prostate | metastatic | 17 | q22 | Hs.298564 | ESTs | |
| 786 | 767 | BG165011 | prostate | metastatic | 17 | q23.2 | Hs.165909 | ESTs Weakly similar to hypothetical protein | |
| | | | | | | | | FLJ20489 [Homo sapiens] [H. sapiens] | |
| 787 | 742 | U28386 | prostate | metastatic | 17 | q24.3 | Hs.159557 | karyopherin alpha 2 (RAG cohort 1 importin | |
| | | | | | | | | alpha 1) | |
| 788 | 713 | K02581 | prostate | metastatic | 17 | q25.3 | Hs.105097 | thymidine kinase 1 soluble | |
| 789 | 719 | AA312511 | prostate | metastatic | 17 | q25.3 | Hs.273307 | signal recognition particle 68 kDa | |
| 790 | 759 | AI525822 | prostate | metastatic | 17 | q25.3 | Hs.109706 | hematological and neurological expressed 1 | |
| 791 | 772 | AI733461 | prostate | metastatic | 18 | p11.22 | Hs.127716 | ESTs | |
| 792 | 783 | AB000277 | prostate | metastatic | 18 | p11.31 | Hs.75814 | discs large (Drosophila) homolog-associated | |
| | | | | | | | | protein 1 | |
| 793 | 712 | X02308 | prostate | metastatic | 18 | p11.32 | Hs.82962 | thymidylate synthetase | |
| 794 | 709 | M91670 | prostate | metastatic | 19 | q13.42 | Hs.174070 | ubiquitin carrier protein | |
| 795 | 708 | AA719022 | prostate | metastatic | 19 | q13.43 | Hs.288549 | ubiquitin UBF-fl | |
| 796 | 792 | AI761506 | prostate | metastatic | 20 | p13 | Hs.274422 | chromosome 20 open reading frame 27 | |
| 797 | 793 | H06350 | prostate | metastatic | 20 | p13 | Hs.135056 | chromosome 20 open reading frame 139 | |
| 798 | 720 | AF011468 | prostate | metastatic | 20 | q13.31 | Hs.250822 | serine/threonine kinase 6 | |
| 799 | 714 | AW016409 | prostate | metastatic | 20 | q13.33 | Hs.235782 | solute carrier family 21 (organic anion | |
| | | | | | | | | transporter) member 12 | |
| 800 | 765 | X70940 | prostate | metastatic | 20 | q13.33 | Hs.2642 | eukaryotic translation elongation factor 1 alpha 2 | |
| 801 | 791 | AI652030 | prostate | metastatic | 21 | q22.11 | Hs.49932 | chromosome 21 open reading frame 45 | |
| 802 | 703 | AI861913 | prostate | metastatic | 21 | q22.3 | Hs.143638 | WD repeat domain 4 | |
| 803 | 757 | AA577678 | prostate | metastatic | 21 | q22.3 | Hs.282961 | Homo sapiens cDNA FLJ35467 fis clone | |
| | | | | | | | | SMINT2005624 | |
| 804 | 781 | AI860822 | prostate | metastatic | 21 | q22.3 | Hs.110757 | DNA segment on chromosome 21 (unique) | |
| | | | | | | | | 2056 expressed sequence | |
| 805 | 790 | AI983544 | prostate | metastatic | 21 | q22.3 | Hs.126522 | chromosome 21 open reading frame 70 | |
| 806 | 120 | R62346 | breast | metastatic | 1 | q23.2 | NULL | unknown | Protein |
| 807 | 120 | R62346 | breast | metastatic | 1 | q23.2 | NULL | unknown | Protein |
| 808 | 120 | R62346 | breast | metastatic | 1 | q23.2 | NULL | unknown | Protein |
| 809 | 120 | R62346 | breast | metastatic | 1 | q23.2 | NULL | unknown | Protein |
| 810 | 120 | R62346 | breast | metastatic | 1 | q23.2 | NULL | unknown | Protein |
| 811 | 120 | R62346 | breast | metastatic | 1 | q23.2 | NULL | unknown | Transcript |
| 812 | 120 | R62346 | breast | metastatic | 1 | q23.2 | NULL | unknown | Transcript |
| 813 | 120 | R62346 | breast | metastatic | 1 | q23.2 | NULL | unknown | Transcript |
| 814 | 120 | R62346 | breast | metastatic | 1 | q23.2 | NULL | unknown | Transcript |
| 815 | 120 | R62346 | breast | metastatic | 1 | q23.2 | NULL | unknown | Transcript |
| 816 | 120 | R62346 | breast | metastatic | 1 | q23.2 | NULL | unknown | Transcript |
| 817 | 120 | R62346 | breast | metastatic | 1 | q23.2 | NULL | unknown | Transcript |
| 818 | 197 | AA663786 | breast | metastatic | 3 | p21.31 | NULL | unknown | Transcript |
| 819 | 194 | AI962335 | breast | metastatic | 3 | p24.3 | Hs.196042 | ESTs | Transcript |
| 820 | 227 | W25552 | breast | metastatic | 9 | q34.3 | Hs.212613 | hypothetical protein FLJ36779 | Protein |
| 821 | 227 | W25552 | breast | metastatic | 9 | q34.3 | Hs.212613 | hypothetical protein FLJ36779 | Transcript |
| 822 | 77 | AI962335 | breast | primary | 3 | p24.3 | Hs.196042 | ESTs | Transcript |
| 823 | 101 | W25552 | breast | primary | 9 | q34.3 | Hs.212613 | hypothetical protein FLJ36779 | Protein |
| 824 | 101 | W25552 | breast | primary | 9 | q34.3 | Hs.212613 | hypothetical protein FLJ36779 | Transcript |
| 825 | 334 | AI962335 | colon | metastatic | 3 | p24.3 | Hs.196042 | ESTs | Transcript |
| 826 | 393 | W25552 | colon | metastatic | 9 | q34.3 | Hs.212613 | hypothetical protein FLJ36779 | Protein |
| 827 | 393 | W25552 | colon | metastatic | 9 | q34.3 | Hs.212613 | hypothetical protein FLJ36779 | Transcript |
| 828 | 301 | AI962335 | colon | primary | 3 | p24.3 | Hs.196042 | ESTs | Transcript |
| 829 | 328 | W25552 | colon | primary | 9 | q34.3 | Hs.212613 | hypothetical protein FLJ36779 | Protein |
| 830 | 328 | W25552 | colon | primary | 9 | q34.3 | Hs.212613 | hypothetical protein FLJ36779 | Transcript |
| 831 | 527 | AK022113 | lung | primary | 1 | p31.3 | Hs.301858 | Homo sapiens cDNA FLJ13017 fis, | Transcript |
| | | | | | | | | clone NT2RP3000628 | |
| 832 | 527 | AK022113 | lung | primary | 1 | p31.3 | Hs.301858 | Homo sapiens cDNA FLJ13017 fis, | Transcript |
| | | | | | | | | clone NT2RP3000628 | |
| 833 | 458 | AA383208 | lung | primary | 5 | p15.1 | Hs.125249 | ESTs | Protein |
| 834 | 458 | AA383208 | lung | primary | 5 | p15.1 | Hs.125249 | ESTs | Transcript |
| 835 | 519 | C00851 | lung | primary | 5 | p13.2 | Hs.144264 | ESTs, Weakly similar to hypothetical | Transcript |
| | | | | | | | | protein FLJ20837 [Homo sapiens] | |
| | | | | | | | | [H. sapiens] | |
| 836 | 505 | AA904882 | lung | primary | 8 | q22.3 | Hs.130107 | ESTs | Transcript |
| 837 | 529 | AK024242 | lung | primary | 8 | q21.11 | Hs.296753 | Homo sapiens cDNA FLJ14180 fis, | Transcript |
| | | | | | | | | clone NT2RP2003799 | |
| 838 | 529 | AK024242 | lung | primary | 8 | q21.11 | Hs.296753 | Homo sapiens cDNA FLJ14180 fis, | Protein |
| | | | | | | | | clone NT2RP2003799 | |
| 839 | 555 | AF232217 | lung | primary | 8 | q13.3 | NULL | unknown | Transcript |
| 840 | 513 | AI146765 | lung | primary | 18 | p11.31 | Hs.373550 | ESTs | Transcript |
| 841 | 753 | N29457 | prostate | metastatic | 1 | q31.1 | Hs.117305 | hypothetical gene supported by | Protein |
| | | | | | | | | BC007071 | |
| 842 | 753 | N29457 | prostate | metastatic | 1 | q31.1 | Hs.117305 | hypothetical gene supported by | Protein |
| | | | | | | | | BC007071 | |
| 843 | 753 | N29457 | prostate | metastatic | 1 | q31.1 | Hs.117305 | hypothetical gene supported by | Protein |
| | | | | | | | | BC007071 | |
| 844 | 753 | N29457 | prostate | metastatic | 1 | q31.1 | Hs.117305 | hypothetical gene supported by | Protein |
| | | | | | | | | BC007071 | |
| 845 | 753 | N29457 | prostate | metastatic | 1 | q31.1 | Hs.117305 | hypothetical gene supported by | Protein |
| | | | | | | | | BC007071 | |
| 846 | 753 | N29457 | prostate | metastatic | 1 | q31.1 | Hs.117305 | hypothetical gene supported by | Transcript |
| | | | | | | | | BC007071 | |
| 847 | 753 | N29457 | prostate | metastatic | 1 | q31.1 | Hs.117305 | hypothetical gene supported by | Transcript |
| | | | | | | | | BC007071 | |
| 848 | 753 | N29457 | prostate | metastatic | 1 | q31.1 | Hs.117305 | hypothetical gene supported by | Protein |
| | | | | | | | | BC007071 | |
| 849 | 753 | N29457 | prostate | metastatic | 1 | q31.1 | Hs.117305 | hypothetical gene supported by | Transcript |
| | | | | | | | | BC007071 | |
| 850 | 753 | N29457 | prostate | metastatic | 1 | q31.1 | Hs.117305 | hypothetical gene supported by | Transcript |
| | | | | | | | | BC007071 | |
| 851 | 753 | N29457 | prostate | metastatic | 1 | q31.1 | Hs.117305 | hypothetical gene supported by | Transcript |
| | | | | | | | | BC007071 | |
| 852 | 753 | N29457 | prostate | metastatic | 1 | q31.1 | Hs.117305 | hypothetical gene supported by | Transcript |
| | | | | | | | | BC007071 | |
| 853 | 753 | N29457 | prostate | metastatic | 1 | q31.1 | Hs.117305 | hypothetical gene supported by | Transcript |
| | | | | | | | | BC007071 | |
| 854 | 753 | N29457 | prostate | metastatic | 1 | q31.1 | Hs.117305 | hypothetical gene supported by | Transcript |
| | | | | | | | | BC007071 | |
| 855 | 705 | AI750154 | prostate | metastatic | 5 | p15.1 | NULL | unknown | Protein |
| 856 | 705 | AI750154 | prostate | metastatic | 5 | p15.1 | NULL | unknown | Transcript |
| 857 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 858 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 859 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 860 | 755 | H04885 | prostate | metastatic | 14 | q32.31 | Hs.72363 | Homo sapiens, clone MGC: 16771 | Protein0 |
| | | | | | | | | IMAGE: 3907551, mRNA, complete cds | |
| 861 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 862 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 863 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 864 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 865 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 866 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 867 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 868 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 869 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 870 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 871 | 755 | H04885 | prostate | metastatic | 14 | q32.31 | Hs.72363 | Homo sapiens, clone MGC: 16771 | Protein0 |
| | | | | | | | | IMAGE: 3907551, mRNA, complete cds | |
| 872 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 873 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 874 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 875 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 876 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 877 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 878 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 879 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 880 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 881 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 882 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 883 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 884 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 885 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 886 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 887 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 888 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 889 | 755 | H04885 | prostate | metastatic | 14 | q32.31 | Hs.72363 | Homo sapiens, clone MGC: 16771 | Protein |
| | | | | | | | | IMAGE: 3907551, mRNA, complete cds | |
| 890 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 891 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 892 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 893 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 894 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 895 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 896 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 897 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 898 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 899 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 900 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 901 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 902 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 903 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 904 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 905 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 906 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 907 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 908 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 909 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 910 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 911 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 912 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 913 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 914 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 915 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 916 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 917 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 918 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 919 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 920 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 921 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 922 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |
| 923 | 755 | H04885 | prostate | metastatic | 14 | q32.31 | Hs.72363 | Homo sapiens, clone MGC: 16771 | Transcript |
| | | | | | | | | IMAGE: 3907551, mRNA, complete cds | |

1-4. (canceled)

5. A method for diagnosing cancer or a pre-cancerous condition in a mammal, comprising:
(a) obtaining a cell or tissue sample from a mammal suspected of having cancer or a pre-cancerous condition and determining for said sample the gene copy number of a gene of Table 1;
(b) comparing said gene copy number of step (a) to the gene copy number of the same gene from a sample of a corresponding cell or tissue from a mammal of the same species not having cancer of the type being diagnosed
whereby a higher gene copy number determined in step (a) relative to that in step (b) indicates the presence of a cancer or pre-cancerous condition in the mammal of step (a) and results in a diagnosis of cancer or a pre-cancerous condition in said mammal.

6. The method of claim 5 wherein said mammal is a human patient.

7. The method of claim 5 wherein said cancer is a member selected from breast cancer, colon cancer, lung cancer, prostate cancer, ovarian cancer, pancreatic cancer, cervical cancer and kidney cancer.

8. The method of claim 5 wherein the gene of Table 1 is a gene that encodes the same gene product as a polynucleotide of SEQ ID NO: 1-805 and 855-923.

9. A method of inhibiting cancer, or a pre-cancerous condition, in a mammalian cell, comprising contacting said cell with a molecule that inhibits function of a gene of Table 1.

10. The method of claim 9 wherein said gene of Table 1 is a gene that encodes the same gene product as a polynucleotide of SEQ ID NO: 1-805 and 855-923.

11. The method of claim 9 wherein said molecule inhibits gene function by binding to said gene.

12. The method of claim 9 wherein said molecule inhibits gene function by binding to an RNA encoded by said gene.

13. The method of claim 9 wherein said molecule inhibits gene function by binding to polypeptide encoded by said gene.

14. The method of claim 9 wherein said molecule is a member selected from an antisense DNA, an antisense RNA, a ribozyme and an siRNA.

15. The method of claim 9 wherein said cancer is a member selected from breast cancer, colon cancer, lung cancer, prostate cancer, ovarian cancer, pancreatic cancer, cervical cancer and kidney cancer.

16. The method of claim 9 wherein said contacting occurs in vivo.

17. A method for identifying an agent having therapeutic activity in a human patient in need of said therapeutic activity, comprising:
(a) determining in a sample from a patient the level of a gene product encoded by a gene of Table 1 prior to administering a test compound to said patient;
(b) administering said test compound to said patient;
(c) determining in a sample from said patient the level of a gene product encoded by the same the gene as in step (a)
wherein a decrease in the level of said gene product in step (c) relative to step (a) identifies said test compound as an agent having therapeutic activity.

18. The method of claim 17 wherein said therapeutic activity is anticancer activity.

19. The method of claim 17 wherein said cancer is a member selected from breast cancer, colon cancer, lung cancer, prostate cancer, ovarian cancer, pancreatic cancer, cervical cancer and kidney cancer.

20. The method of claim 17 wherein said gene product is an RNA.

21. The method of claim 17 wherein said gene product is a polypeptide.

22. The method of claim 21 wherein said determination of said polypeptide is a determination of an enzyme activity.

23. The method of claim 17 wherein said gene of Table 1 is a gene that encodes the same gene product as a polynucleotide of SEQ ID NO: 1-805 and 855-923.

24. The method of claim 17 wherein said molecule is a member selected from an antisense DNA, an antisense RNA, a ribozyme and an siRNA.

25. A method for identifying an antineoplastic agent, comprising:
(a) contacting a test compound with a cell that expresses a gene of Table 1; and
(b) determining a change in gene expression as a result of said contacting;
whereby said change in said gene expression identifies said test compound as an antineoplastic agent.

26-27. (canceled)

28. The method of claim 25 wherein said gene of Table 1 encodes the same gene product as a polynucleotide of SEQ ID NO: 1-805 and 855-923.

29. The method of claim 25 wherein said molecule is a member selected from an antisense DNA, an antisense RNA, ribozyme, an siRNA, a small organic molecule and an antibody.

30. A method for determining the cancerous status of a cell, comprising determining elevated expression in said cell of a gene of Table 1 wherein elevated expression of said gene indicates that said cell is cancerous.

31. (canceled)

32. The method of claim 30 wherein said gene of Table 1 encodes the same gene product as a polynucleotide of SEQ ID NO: 1-805 and 855-923.

33. A method for identifying a compound as an anti-neoplastic agent, comprising:
(a) contacting a test compound with a polypeptide encoded by a gene of Table 1,
(b) determining a change in a biological activity of said polypeptide due to said contacting,
wherein a change in activity identifies said test compound as an agent having antineoplastic activity.

34. The method of claim 33 wherein said gene of Table encodes the same gene product as a polynucleotide of SEQ ID NO: 1-805 and 855-923.

35. (canceled)

36. The method of claim 33 wherein said biological activity is an enzyme activity.

37-55. (canceled)

56. The method of claim 33 wherein said polypeptide is contained in a cell.

57. The method of claim 33 wherein said molecule is a member selected from antisense DNA, an antisense RNA, a ribozyme, an siRNA, a small organic molecule and an antibody.

58. The method of claim 57 wherein said antibody is specific for a polypeptide comprising an amino acid sequence of SEQ ID NO: 806 -854.

59-61. (canceled)

62. A method for treating cancer comprising contacting a cancerous cell with an agent first identified as having gene modulating activity using the method of claim 25, 33, or 58 and in an amount effective to cause a reduction in cancerous activity of said cell.

63-66. (canceled)

67. A method for treating cancer comprising contacting a cancerous cell with an agent having affinity for an expression product of a gene of Table 1 and in an amount effective to cause a reduction in cancerous activity of said cell.

68. The method of claim 67 wherein said expression product is a polypeptide.

69. The method of claim 67 wherein said molecule is a member selected from antisense DNA, an antisense RNA, a ribozyme, an siRNA, a small organic molecule and an antibody.

70. The method of claim 69 wherein said antibody is specific for a polypeptide comprising an amino acid sequence selected from SEQ ID NO: 806-854.

71. A method for monitoring the progress of cancer therapy in a patient comprising monitoring in a patient undergoing cancer therapy the expression of a gene of Table 1.

72. The method of claim 71 wherein said gene encodes the same gene product as a polynucleotide of SEQ ID NO: 1-805 and 855-923.

73-74. (canceled)

75. A method for determining the likelihood of success of cancer therapy in a patient, comprising monitoring in a patient undergoing cancer therapy the expression of a gene of Table 1 wherein a decrease in said expression prior to completion of said cancer therapy is indicative of a likelihood of success of said cancer therapy.

76. The method of claim 75 wherein said gene encodes the same gene product as a polynucleotide of SEQ ID NO: 1-805 and 855-923.

77-78. (canceled)

79. A method for producing test data with respect to the anti-neoplastic activity of a compound comprising:
(a) identifying a test compound as having anti-neoplastic activity using a method of claim 25;
(b) producing test data with respect to the anti-neoplastic activity of said test compound sufficient to identify the chemical structure of said test compound.

80. A method for producing test data with respect to the anti-neoplastic activity of a compound comprising:
(a) identifying a test compound as having anti-neoplastic activity using a method of claim 33;
(b) producing test data with respect to the anti-neoplastic activity of said test compound sufficient to identify the chemical structure of said test compound.

81. A method for determining the progress of a treatment for cancer in a patient afflicted therewith, following commencement of a cancer treatment on said patient, comprising:
(a) determining in said patient a change in expression of one or more genes of Table 1; and
(b) determining a change in expression of said gene compared to expression of said one or more determined genes prior to said cancer treatment;
wherein said change in expression indicates progress of said treatment thereby determining the progress of said treatment.

82. The method of claim 81 wherein said change in expression is a decrease in expression and said decrease indicates success of said treatment.

83. The method of claim 81 wherein said gene encodes the same gene product as a polynucleotide of SEQ ID NO: 1-805 and 855-923.
